(19) United States
(12) Patent Application Publication
(10) Pub. No.: US 2012/0134972 A1
(43) Pub. Date: May 31, 2012

(54) Novel Polypeptides Having Endolysin Activity And Uses Thereof

(75) Inventors: Melinda Mayer, Norfolk (GB); Arjan Narbad, Norfolk (GB)

(73) Assignee: PLANT BIOSCIENCE LIMITED, Norfolk (GB)

(21) Appl. No.: 13/322,204

(22) PCT Filed: May 26, 2010
(86) PCT No.: PCT/GB10/01035

§ 371 Date: Nov. 23, 2011

(30) Foreign Application Priority Data
May 26, 2009 (GB) 0908949.1

(51) Int. Cl. A61K 48/00; C12N 15/55; C12N 15/63; C12N 1/21; C12N 1/19; C12N 1/15; C12N 5/10; A61K 35/76; A61K 38/50; A01N 63/00; A01N 63/02; A61P 31/04; A01P 1/00; C12Q 1/68; C12Q 1/70; C12Q 1/34; C12N 9/80

(52) U.S. Cl. 424/93.21; 435/228; 536/23.2; 435/320.1; 435/252.33; 435/252.31; 435/254.21; 435/254.3; 435/419; 435/325; 435/348; 435/354; 435/353; 435/364; 435/363; 435/369; 435/366; 435/358; 435/357; 435/252.3; 424/93.6; 424/93.2; 514/44 R; 424/94.6; 435/6.15; 435/5; 435/18

(57) ABSTRACT

The present invention provides isolated polypeptides comprising a fragment of the amino acid sequence of SEQ ID NO:1, or a variant, derivative or fusion thereof, which is capable of binding specifically to and lysing cells of Clostridium difficile, wherein the polypeptide exhibits greater lytic activity on cells of Clostridium difficile than the polypeptide of SEQ ID NO: 1. The invention further provides means for producing the same, methods for killing bacterial cells such as cells of Clostridium difficile, as well as methods for diagnosing, treating and preventing diseases and conditions associated with infection of the same.

### FIELD OF INVENTION

The present invention relates to novel polypeptides derived from endolysins from a bacteriophage of Clostridium difficile and nucleic acid molecules encoding the same, as well as compositions thereof. The invention also provides uses of such polypeptides and nucleic acid molecules in the diagnosis and treatment of conditions and diseases associated with microbial cells such as Clostridium difficile. In particular, the invention provides polypeptides comprising a fragment of an endolysin from bacteriophage φCD27 of Clostridium difficile, which polypeptides exhibit improved lytic activity.

### INTRODUCTION

The growing problems associated with Clostridium difficile are well documented, in particular its role in nosocomial infections often associated with antibiotic use (1). C. difficile is an anaerobic Gram positive bacterium that has the capacity to form spores that resist heating, drying and disinfectants. There is some evidence that exposure to non-chlorine based cleaning agents actually increases sporulation. These characteristics contribute the organism's capacity to persist in the hospital environment, thereby maintaining a reservoir of pathogens with the potential to infect patients. C. difficile-associated disease (CDAD) is a growing problem both in the UK and worldwide, with both rates and severity increasing. In England and Wales, deaths associated with C. difficile infection rose from 975 in 1999 to 2,247 in 2004. CDAD notifications rose from 1000 in 1999 to 15,000 in 2000 and 35,500 in 2003 (2). It should be noted that, in addition to threats to human health mentioned above, C. difficile is also a significant cause of morbidity and mortality in animals, particularly in farm animals such as calves and sheep. Accordingly, disclosure herein as to methods for addressing this problem in humans should likewise be read to apply to veterinary targets as well.

A particularly serious development is the emergence of a highly virulent strain of C. difficile, initially in Canada and the USA, but now significant in the UK and several other European countries. This new strain, defined as C. difficile ribotype 027, was detected in the UK in 2003 in an outbreak involving 174 cases and 19 deaths. By April 2006 there have been 450 separate UK isolates of C. difficile ribotype 027 from 75 hospitals (1).

C. difficile is widely distributed in soil and in the intestinal tracts of animals. It can be cultured from the stools of 3% of healthy human adults and 80% of healthy newborns and infants (1). Pathogenic potential is associated with the ability of C. difficile to produce potent toxins; the two major characterised toxins are a 308 kDa exotoxin, toxin A (TcdA) and a 270 kDa cytotoxin, toxin B (TcdB), which share 63% homology at the amino acid level (3). Genes encoding these toxins are associated with a pathogenicity island PaLoc (4) and strains vary in their ability to produce these two major toxins. Other virulence factors are likely to be involved, and a separate binary toxin CDT has been defined (5, 6).

The pathogenic potential of virulent C. difficile strains is realised when the gastro-intestinal tract (GIT) microflora becomes impaired or unbalanced, and this is a common consequence of antibiotic therapy. Thus the hospital environment is an ideal one for C. difficile to thrive and cause human disease (1).

CDAD occurs when pathogenic strains of C. difficile gain a sufficiently strong position within the GIT microflora and produce toxin(s) that damage the host epithelium. The GIT microflora is an important barrier to pathogenic microbes, representing a complex community of some 500 to 1000 different species that are maintained in a homeostatic equilibrium interacting in beneficial ways with the host. Classical antibiotic therapy is variably non-discriminatory and it can damage the fine balance of the GIT microbial community. The disruption of the normal microflora is a major factor in the manifestation of CDAD, either as consequence of prior antibiotic therapy or another factor.

Hence, there exists a growing need for new treatments and approaches for the control of C. difficile without damaging the protective capacity of the complex GIT microflora.

### SUMMARY OF INVENTION

A first aspect of the invention provides an isolated polypeptide comprising a fragment of the amino acid sequence of SEQ ID NO:1, or a variant, derivative or fusion thereof, wherein the polypeptide is capable of binding specifically to and lysing cells of Clostridium difficile and wherein the polypeptide exhibits greater lytic activity on cells of Clostridium difficile than the polypeptide of SEQ ID NO: 1.

The amino acid sequence depicted below is that of the wildtype (i.e. naturally occurring) endolysin (“CD27L”) of bacteriophage φCD27 of Clostridium difficile.

[SEQ ID NO: 1]
MKICITVGHSILKSGACTSADGVVNEYQYNKSLAPVLADTFRKEGHKVDVIICPEKQFKT
KNEEKSYK1PRVNSGGYDLLIELHLNASNGQGKGSEVLYYSNKGLEYATRICDKLGTVFK
NRGAKLDKRLYILNSSKPTAVLIESFFCDNKEDYDKAKKLGHEGIAKLIVEGVLNKNINNE
GVKQMYKHTIVYDGEVDKISATVVGWGYNDGKILICDIKDYVPGQTQNLYVVGGGACEK
ISSITKEKFIMIKGNDRFDTLYKALDFINR

See also NCBI Accession Nos. YP_{—}002290910 and ACH91325.

By “capable of binding specifically to cells of Clostridium difficile” we mean that the polypeptide is capable of binding preferentially to cells of Clostridium difficile. However, it will be appreciated that such polypeptides may also bind preferentially to one or more additional types of cell. Preferably, the polypeptide binds exclusively to cells of Clostridium sp. Such cell binding activity may be determined using methods well known in the art.

A characterising feature of the polypeptides of the present invention is that they exhibit greater lytic activity on cells of Clostridium difficile than the polypeptide of SEQ ID NO: 1.

By “lytic activity” or “capable of lysing cells of Clostridium difficile” we mean that the polypeptide, or fragment, variant, derivative or fusion, retains the ability of the wildtype endolysin of bacteriophage φCD27 to lyse bacterial cells. It will be appreciated that such lytic activity should be cell-specific (e.g. to cells of Clostridium difficile) rather than a non-specific cytotoxic activity on all cell types. Such cell lysis activity may be determined using methods well known in the art, such as those described in detail in the Examples below (see also Loessner et al. [37], the disclosures of which are incorporated herein by reference). In a preferred embodiment, the ability of polypeptides to lyse cells of Clostridium difficile is determined using cells of strain 11204. Preferably, the ability of polypeptides to lyse cells of Clostridium difficile is determined using fresh cells.

By “greater lytic activity” we include that the polypeptide of the invention is able to lyse cells of Clostridium difficile more quickly and/or to a greater extent than the wildtype endolysin of bacteriophage φCD27 (see SEQ ID NO:1 above).

For example, the polypeptide may exhibit at least 110% of the lytic activity of the polypeptide of SEQ ID NO: 1 on cells of Clostridium difficile, for example at least 120%, 130%, 140%, 150%, 160%, 170%, 180%, 200%, 250%, 300%, 400% or 500%.

In one embodiment, the polypeptide of the invention exhibits greater lytic activity on cells of Clostridium difficile ribotype 027 than the polypeptide of SEQ ID NO: 1.

Most preferably, however, the polypeptide exhibits greater lytic activity on all strains of Clostridium difficile than the polypeptide of SEQ ID NO: 1.

In one embodiment, the polypeptide of the invention is at least 50 amino acids in length, for example at least 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450 or 500 amino acids in length.

Likewise, the polypeptide of the invention may be fewer than 500 amino acids in length, for example fewer than 450, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60 or 50 amino acids in length.

In a preferred embodiment, however, the polypeptide is between 50 and 200 amino acids in length, for example, between 100 and 200, between 150 and 200, between 160 and 190 or between 170 and 180 amino acids in length.

The term ‘amino acid’ as used herein includes the standard twenty genetically-encoded amino acids and their corresponding stereoisomers in the ‘D’ form (as compared to the natural form), omega-amino acids and other naturally-occurring amino acids, unconventional amino acids (e.g. α,α-disubstituted amino acids, N-alkyl amino acids, etc.) and chemically derivatised amino acids (see below).

Thus, when an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

Preferably, the polypeptide, or variant, fusion or derivative thereof, comprises or consists of L-amino acids.

By “isolated” we mean that the polypeptide of the invention is provided in a form other than that in which it may be found naturally. Preferably, the polypeptide is provided free from intact bacteriophage.

It will be appreciated that naturally occurring lysins of a bacteriophage of Clostridium difficile known in the prior art are not encompassed by the first aspect of the invention. Thus, the first aspect of the invention provides isolated polypeptides comprising or consisting of a fragment of the amino acid sequence of SEQ ID NO:1 and non-naturally occurring fragments, variants, derivatives or fusions thereof.

In particular, the following lysins of a bacteriophage of Clostridium difficile are explicitly excluded from the scope of the first aspect of the invention:
(a) the lysin of bacteriophage φCD27;
(b) the lysin of bacteriophage φCD119;
(c) the lysin of bacteriophage φC2; and
(d) the lysin of prophages 1 and 2 of Clostridium difficile strain 630 (CD630).

For example, the following known proteins (defined by reference to their NCBI accession numbers) are explicitly excluded from the scope of the first aspect of the invention:

| | |
|---|---|
| PhiCD27 endolysin | YP_002290910 and ACH91325 |
| PhiC2 putative endolysin | YP_001110754 |
| CD630 phage endolysin (prophage 1) | YP_001087453 |
| PhiCD119 putative lysin | YP_529586 |
| QCD-32g58 hypothetical protein | ZP_01803398 |
| QCD-32g58 hypothetical protein | ZP_01803228 |

The polypeptides of the first aspect of the invention comprise a fragment of the amino acid sequence of SEQ ID NO:1, or a variant, derivative or fusion thereof.

By “fragment” we mean part (but not all) of the of the amino acid sequence of SEQ ID NO:1. Thus, the fragment may comprise at least 50 contiguous amino acids of SEQ ID NO: 1, for example at least 60, 70, 80, 90, 100, 110, 120, 130, 140, 150, 160, 170, 180, 190, 200, 210, 220, 230, 240 or 250 contiguous amino acids of SEQ ID NO: 1.

In one embodiment, the N-terminus of the fragment corresponds to amino acid 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 49, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 149, 147, 148, 149 or 150 of SEQ ID NO:1.

In a further embodiment, the C-terminus of the fragment corresponds to amino acid 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 149, 147, 148, 149, 150, 151, 152, 153, 154, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237, 238, 239, 240, 241, 242, 243, 244, 245, 246, 249, 247, 248, 249, 250, 251, 252, 253, 254, 256, 257, 258, 259, 260, 261, 262, 263, 264, 265, 266, 267, 268, 269 or 270 of SEQ ID NO:1.

Advantageously, the fragment corresponds to amino acids 1 to 179 of SEQ ID NO:1 (i.e. CD27L₁₋₁₇₉; corresponding to truncated endolysin “27*” in Examples; see SEQ ID NO:2 below).

[SEQ ID NO: 2]
MKICITVGHSILKSGACTSADGVVNEYQYNKSLAPVLADTFRKEGHKVDVIICPEKQFKT
KNEEKSYKIPRVNSGGYDLLIELHLNASNGQGKGSEVLYYSNKGLEYATRICDKLGTVFK
NRGAKLDKRLYILNSSKPTAVLIESFFCDNKEDYDKAKKLGHEGIAKLIVEGVLNKNIN

Thus, it will be appreciated by skilled persons that the polypeptides of the invention may comprise or consist of a fragment of the amino acid sequence of SEQ ID NO:1, or a variant, derivative or fusion of such a fragment.

It is well established that many bacteriophage endolysins consist of two distinct domains (for example, see Sheehan et al., 1996, FEMS Microbiology Letters 140:23-28, the disclosures of which are incorporated herein by reference). One is a catalytic domain that is responsible for cell wall degradation and these are known to exist in several different forms. The other domain is a cell wall binding domain that recognises a cell surface motif and permits attachment of the endolysin to that target cell. The precise pattern recognition involved in the latter is what provides the specificity.

The polypeptides of the first aspect of the invention preferably comprise or consist of one or more fragments of the amino acid sequence of SEQ ID NO:1 corresponding to both the enzymatic domain and the cell wall binding domain. However, it will be appreciated by persons skilled in the art that the enzymatic (lytic) domain of SEQ ID NO:1 may alternatively be fused or otherwise coupled to a cell wall binding domain from another source capable of binding and/or lysing cells of Clostridium difficile, or vice-versa. The production of chimeric lysins is described in Sheehan et al., 1996, FEMS Microbiology Letters 140:23-28, the disclosures of which are incorporated herein by reference).

In addition to fragments of the amino acid sequence of SEQ ID NO:1, the first aspect of the invention also extends to variants, derivatives and fusions of such fragments which are capable of binding specifically to cells of Clostridium difficile and which exhibit increased lytic activity.

Thus, in an alternative embodiment, the polypeptide of the first aspect of the invention may comprise or consist of a variant of the amino acid sequence of SEQ ID NO:1, or of a fragment thereof, which is capable of lysing cells of Clostridium difficile.

By ‘variant’ of the polypeptide we include insertions, deletions and/or substitutions, either conservative or non-conservative, relative to the amino acid sequence of the fragment of SEQ ID NO:1. In particular, the variant polypeptide may be a non-naturally occurring variant.

For example, the polypeptide may comprise an amino acid sequence with at least 60% identity to the fragment of the amino acid sequence of SEQ ID NO: 1, more preferably at least 70% or 80% or 85% or 90% identity to said sequence, and most preferably at least 95%, 96%, 97%, 98% or 99% identity to said amino acid sequence.

It will be appreciated that the above sequence identity may be over the full length of the fragment or over a portion thereof. Preferably, however, the sequence identity is over at least 50 amino acids of the amino acid sequence of SEQ ID NO: 1, for example at least 60, 70, 80 90, 100, 110, 120, 130, 140, 150, 160, 170, 180 190, 200, 210, 220, 230, 240, 250, 260 or more amino acids therein.

Percent identity can be determined by methods well known in the art, for example using the LALIGN program (Huang and Miller, Adv. Appl. Math. (1991) 12:337-357, the disclosures of which are incorporated herein by reference) at the ExPASy facility website:
www.ch.embnet.org/software/LALIGN form.html
   using as parameters the global alignment option, scoring matrix BLOSUM62, opening gap penalty −14, extending gap penalty −4.

Alternatively, the percent sequence identity between two polypeptides may be determined using suitable computer programs, for example AlignX, Vector NTI Advance 10 (from Invitrogen Corporation) or the GAP program (from the University of Wisconsin Genetic Computing Group).

It will be appreciated that percent identity is calculated in relation to polypeptides whose sequence has been aligned optimally.

In one embodiment, the variant comprises one or more substitutions of a cysteine residue in the amino acid sequence of SEQ ID NO:1. Thus, the variant may comprise a substitution at a cysteine residue selected from the group consisting of residues 4, 17, 53, 112, 148, 217 and 238 of the amino acid sequence of SEQ ID NO:1. Advantageously, the cysteine residue is selected from the group consisting of residues 53, 217 and 238 of the amino acid sequence of SEQ ID NO:1.

Advantageously, the polypeptide of the invention comprises or consists of a fragment corresponding to amino acids 1 to 179 of SEQ ID NO:1, in which one or more of cysteine residues 4, 17, 53, 112, 148, 217 and 238 is substituted (e.g. for serine).

A further aspect of the invention provides an isolated polypeptide comprising a variant of the amino acid sequence of SEQ ID NO:1, or a fragment, derivative or fusion thereof which is capable of binding specifically to and/or lysing cells of Clostridium difficile, wherein one or more of the cysteine residues of the amino acid sequence of SEQ ID NO:1 is substituted for a different amino acid residue in the polypeptide.

The different amino acid residue (substituted for cysteine) may be any other naturally occurring or non-naturally occurring amino acid, for example serine.

Advantageously, the above-mentioned cysteine substitution mutants exhibit reduced oligomerisation in vitro compared to the amino acid sequence of SEQ ID NO:1. Such mutants may be particularly suited to production using recombinant expression systems, where oligomerisation can be undesirable.

Fragments and variants of the amino acid sequence of SEQ ID NO: 1 may be made using the methods of protein engineering and site-directed mutagenesis well known in the art (for example, see Molecular Cloning: a Laboratory Manual, 3rd edition, Sambrook & Russell, 2001, Cold Spring Harbor Laboratory Press, the disclosures of which are incorporated herein by reference). For example, substitution variants can be made using splice-overlap PCR and other site-directed mutagenesis methods (see Examples below).

It will be appreciated by skilled persons that the polypeptide of the invention may comprise one or more amino acids that are modified or derivatised. Thus, the polypeptide may comprise or consist of a derivative of the fragment of the amino acid sequence of SEQ ID NO:1, or of a variant thereof.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

For example, the polypeptides described herein include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, the disclosures of which are incorporated herein by reference. Such retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -γ(CH₂NH)— bond in place of the conventional amide linkage.

It will be appreciated that the polypeptide may conveniently be blocked at its N- or C-terminus so as to help reduce susceptibility to exoproteolytic digestion, e.g. by amidation.

As discussed above, a variety of uncoded or modified amino acids such as D-amino acids and N-methyl amino acids may be used to modify polypeptides of the invention. In addition, a presumed bioactive conformation may be stabilised by a covalent modification, such as cyclisation or by incorporation of lactam or other types of bridges. Methods of synthesis of cyclic homodetic peptides and cyclic heterodetic peptides, including disulphide, sulphide and alkylene bridges, are disclosed in U.S. Pat. No. 5,643,872. Other examples of cyclisation methods are discussed and disclosed in U.S. Pat. No. 6,008,058, the relevant disclosures in which documents are hereby incorporated by reference. A further approach to the synthesis of cyclic stabilised peptidomimetic compounds is ring-closing metathesis (RCM).

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification and is preferred because of the stable structures formed by cyclisation and in view of the biological activities observed for cyclic peptides.

Thus, in one embodiment the polypeptide, or fragment, variant, fusion or derivative thereof, is cyclic. However, in a preferred embodiment, the polypeptide, or fragment, variant, fusion or derivative thereof, is linear.

In a further embodiment of the first aspect of the invention, the polypeptide comprises or consists of a fusion of the fragment of the amino acid sequence of SEQ ID NO:1, or of a variant or derivative thereof.

By ‘fusion’ of a polypeptide we include a polypeptide which is fused to any other polypeptide. Thus, the polypeptide may comprise one or more additional amino acids, inserted internally and/or at the N- and/or C-termini of the fragment of the amino acid sequence of SEQ ID NO:1, or of a variant or derivative thereof.

For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any fragment, variant or derivative of said polypeptide are also included in the scope of the invention. It will be appreciated that fusions (or variants or derivatives thereof) which retain desirable properties, namely endolysin activity are preferred. It is also particularly preferred if the fusions are ones which are suitable for use in the methods described herein.

In one embodiment, the said polypeptide comprises an oligo-histidine tag (“His tag”), which may be located at its N terminus or C terminus.

For example, the His tag of SEQ ID NO: 3 is particularly suited for expression of the polypeptides of the invention in E. coli (e.g. from vector pET15b):

| | |
|---|---|
| MGSSHHHHHHSSGLVPRGSH | [SEQ ID NO: 3] |

Thus, in one preferred embodiment, the polypeptide comprises or consists of the above His tag fused to the amino terminus of SEQ ID NO:2:

[SEQ ID NO: 4]
MGSSHHHHHHSSGLVPRGSHMKICITVGHSILKSGACTSADGVVNEYQYNKSLAPVLA
DTFRKEGHKVDVIICPEKQFKTKNEEKSYKIPRVNSGGYDLLIELHLNASNGQGKGSEVL
YYSNKGLEYATRICDKLGTVFKNRGAKLDKRLYILNSSKPTAVLIESFFCDNKEDYDKAK
KLGHEGIAKLIVEGVLNKNIN

Similarly, the His tag of SEQ ID NO: 5 is particularly suited for expression of the polypeptides of the invention in L. lactis (e.g. from vector pUK200 or pTG262):

| | |
|---|---|
| MSHHHHHHA | [SEQ ID NO: 5] |

Thus, in a further preferred embodiment, the polypeptide comprises or consists of the above His tag fused to the amino terminus of SEQ ID NO:2:

[SEQ ID NO: 6]
MSHHHHHHAMKICITVGHSILKSGACTSADGVVNEYQYNKSLAPVLADTFRKEGHKVDV
IICPEKQFKTKNEEKSYKIPRVNSGGYDLLIELHLNASNGQGKGSEVLYYSNKGLEYATRI
CDKLGTVFKNRGAKLDKRLYILNSSKPTAVLIESFFCDNKEDYDKAKKLGHEGIAKLIVEG
VLNKNIN

It will be appreciated that the fusion may alternatively or additionally comprise a further portion which confers a desirable feature on the said polypeptide of the invention; for example, the portion may be useful in detecting or isolating the polypeptide, promoting cellular uptake of the polypeptide, or directing secretion of the protein from a cell. The portion may be, for example, a signal peptide, biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that the polypeptides of the invention also include pharmaceutically acceptable acid or base addition salts of the above described polypeptides. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The polypeptide, or fragment, variant, fusion or derivative thereof, may also be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

An essential feature of the polypeptides of the invention is the ability to lyse cells of Clostridium difficile. Preferably, the polypeptide is capable of lysing cells of multiple strains of Clostridium difficile. For example, the polypeptide may be capable of lysing one or more of the strains of Clostridium difficile lysed by the φCD27 lysin of SEQ ID NO: 1 (see Table 1 below).

It will be appreciated that the polypeptides of the invention may also be capable of lysing cells of other bacterial species, such as Bacillus sp. (e.g. Bacillus cereus, Bacillus subtilis and/or Bacillus anthracis), other Clostridium sp. (e.g. Clostridium bifermentans) and/or Listeria sp. (e.g. Listeria ivanovii).

In one embodiment, the polypeptides of the invention are substantially incapable of lysing bacteria which are useful for maintaining a healthy gut physiology, such as members of the Bifidobacteria and Lactobacilli, (see Servin, 2004, FEMS Mircobiology Reviews 28:405-440).

Alternatively, or in addition, it may be advantageous if the polypeptide does not lyse cells of Clostridium leptum, Clostridium nexile, Clostridium coccoides, Clostridium innocuum, Clostridium ramosum, and/or Anaerococcus hydrogenalis.

Most preferably, the polypeptide of the invention is capable of lysing cells of Clostridium difficile strain ribotype 027, a highly virulent strain of Clostridium difficile which has emerged in Canada, the US and now throughout Europe. For example, the polypeptide may exhibit at least 110% of the lysis activity of the polypeptide of SEQ ID NO: 1 on cells of Clostridium difficile ribotype 027, for example at least 120%, 130%, 140%, 150%, 160%, 170%, 180%, 190%, 200%, 250%, 300%, 500% or more.

Advantageously, the polypeptide is capable of lysing cells of pathogenic bacteria selectively, i.e. to a greater extent than cells of non-pathogenic bacteria.

Methods for the production of polypeptides, or a fragment, variant, fusion or derivative thereof, for use in the first aspect of the invention are well known in the art. Conveniently, the polypeptide, or fragment, variant, fusion or derivative thereof, is or comprises a recombinant polypeptide.

Thus, a nucleic acid molecule (or polynucleotide) encoding the polypeptide, or fragment, variant, fusion or derivative thereof, may be expressed in a suitable host and the polypeptide obtained therefrom. Suitable methods for the production of such recombinant polypeptides are well known in the art (for example, see Sambrook & Russell, 2000, Molecular Cloning, A Laboratory Manual, Third Edition, Cold Spring Harbor, N.Y., the relevant disclosures in which document are hereby incorporated by reference).

In brief, expression vectors may be constructed comprising a nucleic acid molecule which is capable, in an appropriate host, of expressing the polypeptide encoded by the nucleic acid molecule.

A variety of methods have been developed to operably link nucleic acid molecules, especially DNA, to vectors, for example, via complementary cohesive termini. For instance, complementary homopolymer tracts can be added to the DNA segment to be inserted into the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, e.g. generated by endonuclease restriction digestion, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities.

The combination of these activities therefore generates blunt-ended DNA segments. The blunt-ended segments are then incubated with a larger molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

The DNA (or in the case of retroviral vectors, RNA) is then expressed in a suitable host to produce a polypeptide. Thus, the DNA encoding the polypeptide may be used in accordance with known techniques, appropriately modified in view of the teachings contained herein, to construct an expression vector, which is then used to transform an appropriate host cell for the expression and production of the compound of the invention or binding moiety thereof. Such techniques are well known in the art.

The DNA (or in the case of retroviral vectors, RNA) encoding the polypeptide may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the expression vector are then cultured for a sufficient time and under appropriate conditions known to those skilled in the art in view of the teachings disclosed herein to permit the expression of the polypeptide, which can then be recovered.

Many expression systems are known, including bacteria (for example, E. coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Other vectors and expression systems are well known in the art for use with a variety of host cells.

The host cell may be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No. ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CRL 1658 and 293 cells which are human embryonic kidney cells. Preferred insect cells are Sf9 cells which can be transfected with baculovirus expression vectors.

Methods of cultivating host cells and isolating recombinant proteins are well known in the art. It will be appreciated that, depending on the host cell, the polypeptides of the invention produced may differ. For example, certain host cells, such as yeast or bacterial cells, either do not have, or have different, post-translational modification systems which may result in the production of forms of compounds of the invention which may be post-translationally modified in a different way.

Polypeptides of the invention may also be produced in vitro using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

Automated polypeptide synthesisers may also be used, such as those available from CS Bio Company Inc, Menlo Park, USA.

Thus, a second aspect of the present invention provides an isolated nucleic acid molecule encoding a polypeptide according to the first aspect of the invention.

The nucleic acid molecule may be DNA (e.g. cDNA) or RNA.

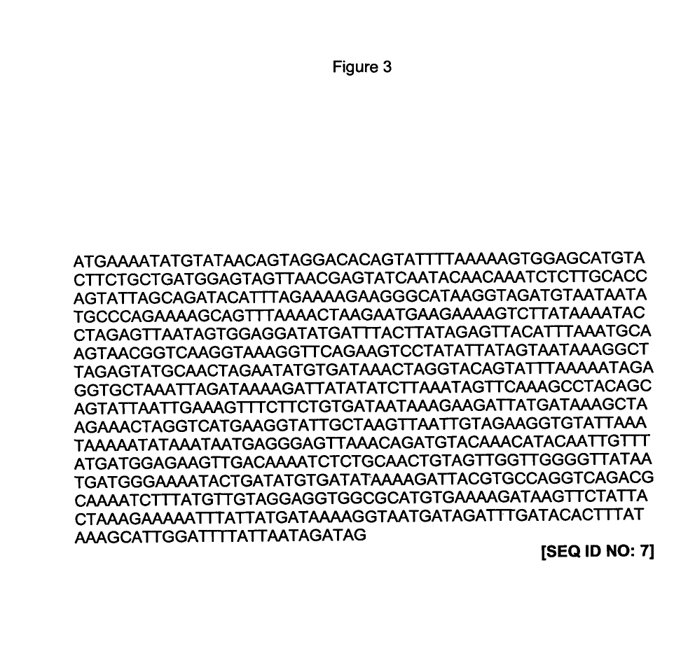

In a preferred embodiment, the nucleic acid molecule comprises or consists of the nucleotide sequence as shown in FIG. 3 [SEQ ID NO:7].

A third aspect of the invention provides a vector comprising a nucleic acid molecule according to the second aspect of the invention. In one embodiment, the vector is an expression vector. Preferably, the vector is selected from the group consisting of pET15b and pACYC184.

It will be appreciated by persons skilled in the art that the choice of expression vector may be determined by the choice of host cell. Thus, for expression of the polypeptides of the invention in Lactococcus lactis, the nisin expression system could be used in which the polypeptide of the invention is expressed under the control of the promoter of the nisA operon using a background strain of Lactococcus lactis which also expresses the nisR and nisK genes encoding a two component regulatory system. Under this system expression is positively regulated and induced by the provision of exogenous nisin (see de Ruyter at el., 1996, Applied and Environmental Microbiology 62:3662-3667, the disclosures of which are incorporated herein by reference).

In an alternative embodiment, the entire nisin biosynthesis gene cluster is provided within the same host cell, in which case the inducer is synthesised by that cell.

In a further alternative embodiment, the polypeptides of the invention may be expressed in Lactococcus lactis under the control of the lactose catabolic operon, using either a plasmid-based or chromasomally integrated system (for example, see Payne et al., 1996, FEMS Microbiology Letters 136: 19-24 and van Rooijen et al., 1992, Journal of Bacteriology 174: 2273-2280, the disclosures of which are incorporated herein by reference).

A fourth aspect of the invention provides a host cell comprising a nucleic acid molecule according to the second aspect of the invention or a vector according to the third aspect of the invention. In one embodiment, the host cell is a microbial cell, for example a bacterial cell. Preferably, the host cell is non-pathogenic.

For example, the host cell may be selected from the group consisting of cells of Escherichia coli, Lactococcus sp., Bacteroides sp., Lactobacillus sp., Enterococcus sp. and Bacillus sp.

In one preferred embodiment, the host cell is a cell of Lactococcus lactis.

In a further preferred embodiment, the host cell is a cell of Lactobacillus johnsonii, for example Lactobacillus johnsonii FI9785.

Alternatively, the host cell may be a yeast cell, for example Saccharomyces sp.

A fifth aspect of the invention provides a method for producing a polypeptide of the invention comprising culturing a population of host cells comprising a nucleic acid molecule according to the second aspect of the invention or a vector according to the third aspect of the invention under conditions in which the polypeptide is expressed, and isolating the polypeptide therefrom.

A sixth aspect of the invention provides a pharmacological composition comprising:
(a) a polypeptide according to the first aspect of the invention;
(b) a nucleic acid molecule according to the second aspect of the invention;
(c) a vector according to the third aspect of the invention;
(d) a host according to the fourth aspect of the invention; and/or
(e) a bacteriophage capable of expressing a polypeptide according to the first aspect of the invention
   and a pharmaceutically acceptable carrier, diluent or excipient.

As used herein, ‘pharmaceutical composition’ means a therapeutically effective formulation for use in the methods of the invention.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

In one embodiment of the invention, the pharmacological composition comprises a polypeptide according to the first aspect of the invention.

The polypeptides can be formulated at various concentrations, depending on the efficacy/toxicity of the polypeptide being used. Preferably, the formulation comprises the polypeptide at a concentration of between 0.1 μM and 1 mM, more preferably between 1 μM and 100 μM, between 5 μM and 50 μM, between 10 μM and 50 μM, between 20 μM and 40 μM and most preferably about 30 μM. For in vitro applications, formulations may comprise similar concentrations of a polypeptide (however, it will be appreciated that higher concentrations may also be used).

Thus, the pharmaceutical formulation may comprise an amount of a polypeptide, or fragment, variant, fusion or derivative thereof, sufficient to inhibit at least in part the growth of cells of Clostridium difficile in a patient who is infected or susceptible to infection with such cells. Preferably, the pharmaceutical formulation comprises an amount of a polypeptide, or fragment, variant, fusion or derivative thereof, sufficient to kill cells of Clostridium difficile in the patient.

It will be appreciated by persons skilled in the art that the polypeptides of the invention are generally administered in admixture with a suitable pharmaceutical excipient, diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, the relevant disclosures in which document are hereby incorporated by reference).

For example, the polypeptides can be administered orally, buccally or sublingually in the form of tablets, capsules, ovules, elixirs, solutions or suspensions, which may contain flavouring or colouring agents, for immediate-, delayed- or controlled-release applications. The polypeptides may also be administered via direct injection (for example, into the GI tract).

Preferably, however, the polypeptides and pharmaceutical compositions thereof are for oral administration.

Suitable tablet formulations may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxyl-propylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the polypeptides may be combined with various sweetening or flavouring agents, colouring matter or dyes, with emulsifying and/or suspending agents and with diluents such as water, ethanol, propylene glycol and glycerin, and combinations thereof.

The polypeptides can also be administered parenterally, for example, intravenously, intra-articularly, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the polypeptides will usually be from 1 to 1000 mg per adult (i.e. from about 0.015 to 15 mg/kg), administered in single or divided doses. For example, a dose of 1 to 10 mg/kg may be used, such as 3 mg/kg.

In an alternative embodiment of the invention, the pharmaceutical compositions do not comprise the polypeptide itself but instead comprise a nucleic acid molecule capable of expressing said polypeptide. Suitable nucleic acid molecules, expression vectors, and host cells are described in detail above.

For example, a recombinant probiotic may be used (LAB strain, e.g. Lactococcus lactis or a Lactobacillus sp.).

In a further embodiment of the invention, the pharmaceutical compositions comprise a bacteriophage capable of expressing a polypeptide according to the first aspect of the invention. For example, the wildtype bacteriophage φCD27 may be used to deliver a polypeptide according to the first aspect of the invention. Methods for performing such bacteriophage-based therapies are well known in the art (for example, see Watanabe et al., 2007, Antimicrobial Agents & Chemotherapy 51:446-452).

Thus, for treatment of bacterial infections described herein, the polypeptide of the invention may be administered as the cognate protein, as a nucleic acid construct, vector or host cell which expresses the cognate protein, as part of a living organism which expresses the cognate protein (including bacteriophages), or by any other convenient method known in the art so as to achieve contact of the lysin with its bacterial target, whether that be a pathogenic bacterium, such as C. difficile, or another pathogen or potential pathogen, as further described herein.

Ideally, the protein is delivered to the GI tract in a protected form. This may be achieved by a wide variety of methods known in the art. For example, an appropriate dose of the lysin is microencapsulated in a form that survives the acidic conditions of the stomach, but which releases the protein as it enters the intestine. Delivery by a non-pathogenic microbe which survives GI tract transit, including but not limited to by Lactococcus lactis, Lactobacillus sp., Bifidobacterium sp. or Bacteroides. Those skilled in the art are well aware of the options available for use of such means for GI tract delivery of active compounds such as the lysin disclosed herein. These means include intracellular production, secA secretion or secretion by means of another secretion pathway, and delivery by controlled lysis. Preferably the protein is not all released at one time, but is released increasingly as an administered bolus traverses through the GI tract. Alternatively, the lysin is introduced as part of a benign bacterium which expresses the lysin at the appropriate location or upon receipt of an appropriate signal in the GI tract. In a preferred embodiment disclosed herein, a non-pathogenic Lactococcus is engineered to express the φCD27 lysin upon reaching a particular location in the GI tract. The expression signal may be defined by a pH sensitive promoter, or another means known in the art for this purpose.

Other means of delivery include the following:
(a) WO 2006/111553 (polyurea and other multilayer encapsulants);
(b) WO 2006/111570 and EP 1 715 739 (cyclodextrin encapsulation);
(c) WO 2006/100308 and EP 1 742 728 (for yeast and other microbial cell encapsulation technologies);
(d) U.S. Pat. No. 5,153,182, EP 1 499 183 and WO 03/092378; U.S. Pat. No. 6,831,070 (therapeutic gene product delivery by intestinal cell expression);
(e) U.S. Pat. No. 7,202,236 (pharmaceutical formulation for modified release);
(f) U.S. Pat. No. 5,762,904 (oral delivery of vaccines using polymerized liposomes, which may be modified to deliver the lysin of this invention),
(g) U.S. Pat. No. 7,195,906 (Bifidobacterium which may be modified to express the lysin according to this invention); and
(h) references cited therein,
   all of which are herein incorporated by reference for purposes of enabling those skilled in the art to utilize the present disclosure to achieve the novel methods of delivery and compositions according to the present invention.

Thus, in a preferred embodiment of the pharmacological compositions of the invention, the polypeptide, nucleic acid molecule encoding the same, etc. is microencapsulated (e.g. within a stable chemical envelope, such as cyclodextrin or a lipid bilayer, or within a living or non-living microbial cell, such as an engineered Lactococcus cell). In this way, the polypeptide, nucleic acid molecule, etc. may be protected against acidic conditions of stomach en route to its site of action in the GI tract.

A seventh aspect of the invention provides a polypeptide according to the first aspect of the invention or pharmacological composition according to the sixth aspect of the invention for use in medicine.

An eighth aspect of the invention provides the use of a polypeptide of the first aspect of the invention, or a nucleic acid molecule, vector, host cell or bacteriophage capable of expressing the same, in the preparation of a medicament for killing and/or inhibiting/preventing the growth of microbial cells in a patient, wherein the microbial cells are selected from the group consisting of Clostridium difficile cells and other bacterial cells susceptible to lysis with the endolysin of SEQ ID NO:1.

A related aspect of the invention provides the use of a polypeptide of the first aspect of the invention, or a nucleic acid molecule, vector, host cell or bacteriophage capable of expressing the same, for killing and/or inhibiting/preventing the growth of microbial cells in a patient, wherein the microbial cells are selected from the group consisting of Clostridium difficile cells and other bacterial cells susceptible to lysis with the endolysin of SEQ ID NO:1.

A further aspect of the invention provides the use of a polypeptide of the first aspect of the invention, or a nucleic acid molecule, vector, host cell or bacteriophage capable of expressing the same, in the preparation of a medicament for the treatment or prevention of a disease or condition associated with microbial cells in a patient, wherein the microbial cells are selected from the group consisting of Clostridium difficile cells and other bacterial cells susceptible to lysis with the endolysin of SEQ ID NO:1. A related aspect of the invention provides the use of a polypeptide of the first aspect of the invention for the treatment or prevention of a disease or condition associated with microbial cells in a patient, wherein the microbial cells are selected from the group consisting of Clostridium difficile cells and other bacterial cells susceptible to lysis with the endolysin of SEQ ID NO:1.

By “a disease or condition associated with microbial cells in a patient” we include diseases and conditions arising from or antagonised by infection of a patient with Clostridium difficile. Such diseases and conditions include Clostridium difficile-associated disease (CDAD).

In one embodiment of the above defined uses of the invention, the microbial cells are selected from the group consisting of Clostridium difficile cells and other bacterial cells susceptible to lysis upon contact with a polypeptide of SEQ ID NO: 1 (see Tables 1 and 2, below).

Preferably, the microbial cells comprise or consist of Clostridium difficile cells. Thus, the polypeptides having the cell lysing activity of an endolysin from a bacteriophage of Clostridium difficile may be used to treat or prevent diseases and conditions associated with infection with Clostridium difficile cells (such as Clostridium difficile-associated disease, CDAD).

Most preferably, the microbial cells comprise or consist of cells are Clostridium difficile ribotype 027 cells.

Thus, the invention further provides the following:
(a) a method for killing and/or inhibiting/preventing the growth of microbial cells in a patient, the method comprising administering to the patient a polypeptide of the first aspect of the invention, or a nucleic acid molecule, vector, host cell or bacteriophage capable of expressing the same, wherein the microbial cells are selected from the group consisting of Clostridium difficile cells and other bacterial cells susceptible to lysis with the endolysin of SEQ ID NO:1;
(b) a method for the treatment or prevention a disease or condition associated with microbial cells in a patient, the method comprising administering to the patient a polypeptide of the first aspect of the invention, or a nucleic acid molecule, vector, host cell or bacteriophage capable of expressing the same, wherein the microbial cells are selected from the group consisting of Clostridium difficile cells and other bacterial cells susceptible to lysis with the endolysin of SEQ ID NO:1.

In one embodiment of the above defined methods of the invention, the microbial cells are selected from the group consisting of Clostridium difficile cells and other bacterial cells susceptible to lysis upon contact with a polypeptide of SEQ ID NO: 1 (see Tables 1 and 2, below). Preferably, the microbial cells comprise or consist of Clostridium difficile cells. Thus, the polypeptide of the first aspect of the invention may be used to treat or prevent diseases and conditions associated with infection with Clostridium difficile cells (such as Clostridium difficile-associated disease, CDAD). Most preferably, the microbial cells comprise or consist of cells of Clostridium difficile ribotype 027.

Persons skilled in the art will further appreciate that the uses and methods of the present invention have utility in both the medical and veterinary fields. Thus, the medicaments may be used in the treatment of both human and non-human animals (such as horses, cows, dogs and cats). Preferably, however, the patient is human.

By ‘treatment’ we include both therapeutic and prophylactic treatment of the patient. The term ‘prophylactic’ is used to encompass the use of a polypeptide or formulation described herein which either prevents or reduces the likelihood of infection with Clostridium difficile in a patient or subject.

As discussed above, the term ‘effective amount’ is used herein to describe concentrations or amounts of polypeptides according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated.

It will be appreciated that the medicaments described herein may be administered to patients in combination with one or more additional therapeutic agents.

For example, the medicaments described herein may be administered to patients in combination with:
(a) one or more conventional antibiotic treatments (such as beta-lactams, aminoglycosides and/or quinolones);
(b) one or more additional lysins, or nucleic acid molecules, vectors, host cell or bacteriophage capable of expressing the same;
(c) one or more lantibiotics, or nucleic acid molecules, vectors, host cell or bacteria capable of expressing the same; and/or
(d) a therapy to neutralise the toxins released upon bacterial lysis of Clostridium difficile cells within the gut. Suitable neutralising therapies may include antibodies (see Babcock et al., 2006, Infect. Immun. 74:6339-6347) and toxin-absorbing agents such as tolevamer (see Barker et al., 2006, Aliment. Pharmacol. Ther. 24:1525-1534).

A further aspect of the invention provides the use of a polypeptide of the first aspect of the invention, or a nucleic acid molecule, vector, host cell or bacteriophage capable of expressing the same, for killing and/or inhibiting/preventing the growth of microbial cells in vitro and/or ex vivo, wherein the microbial cells are selected from the group consisting of Clostridium difficile cells and other bacterial cells susceptible to lysis with the endolysin of SEQ ID NO:1. For example, said polypeptides having endolysin activity may be used to clean surfaces, such as those in hospitals, kitchens, etc, which may be susceptible to contamination with such bacterial cells.

Preferably, the microbial cells are selected from the group consisting of Clostridium difficile cells and other bacterial cells susceptible to lysis upon contact with a polypeptide of SEQ ID NO: 1 (see Tables 1 and 2, below). For example, the microbial cells may comprise or consist of Clostridium difficile cells. Most preferably, the microbial cells comprise or consist of cells of Clostridium difficile ribotype 027.

A further aspect of the present invention provides a kit for detecting the presence of microbial cells in a sample, the kit comprising a polypeptide of the first aspect of the invention, or a nucleic acid molecule, vector, host cell or bacteriophage capable of expressing the same, wherein the microbial cells are selected from the group consisting of Clostridium difficile cells and other bacterial cells susceptible to lysis with the endolysin of SEQ ID NO:1.

In a preferred embodiment, the microbial cells are selected from the group consisting of Clostridium difficile cells and other bacterial cells susceptible to lysis upon contact with a polypeptide of SEQ ID NO: 1 (see Tables 1 and 2, below). For example, the microbial cells may comprise or consist of Clostridium difficile cells. Most preferably, the microbial cells comprise or consist of cells of Clostridium difficile ribotype 027.

In a further embodiment of the kits of the invention, the polypeptide of the first aspect of the invention is immobilised on a suitable surface, such as the surface of a multi-well plate.

The kits may be used in conjunction with any suitable sample of cells, such as tissue samples, cell culture samples and samples of cells derived from swabs (e.g. taken from a surface to be tested for contamination with microbial cells).

Optionally, the kit further comprises a negative control sample (which does not contain cells of the type to be tested for, e.g. Clostridium difficile cells) and/or a positive control sample (which contains cells of the type to be tested for).

Related aspects of the invention provide:
(a) the use of a polypeptide of the first aspect of the invention, or a nucleic acid molecule, vector, host cell or bacteriophage capable of expressing the same, in the preparation of a diagnostic agent for a disease or condition associated with microbial cells selected from the group consisting of Clostridium difficile cells and other bacterial cells susceptible to lysis with the endolysin of SEQ ID NO:1;
(b) the use of a polypeptide of the first aspect of the invention, or a nucleic acid molecule, vector, host cell or bacteriophage capable of expressing the same, for the diagnosis of a disease or condition associated with microbial cells selected from the group consisting of Clostridium difficile cells and other bacterial cells susceptible to lysis with the endolysin of SEQ ID NO:1;
(c) the use of a polypeptide of the first aspect of the invention, or a nucleic acid molecule, vector, host cell or bacteriophage capable of expressing the same, for detecting the presence of microbial cells in a sample in vitro and/or ex vivo, wherein the microbial cells selected from the group consisting of Clostridium difficile cells and other bacterial cells susceptible to lysis with the endolysin of SEQ ID NO:1; and
(d) a method for the diagnosis of a disease or condition associated with microbial cells in a patient, the method comprising contacting a cell sample from a patient to be tested with a polypeptide of the first aspect of the invention, or a nucleic acid molecule, vector, host cell or bacteriophage capable of expressing the same, and determining whether the cells in the sample have been lysed thereby, wherein the microbial cells are selected from the group consisting of Clostridium difficile cells and other bacterial cells susceptible to lysis with said endolysin.

In one embodiment of the above defined uses and methods of the invention, the microbial cells are selected from the group consisting of Clostridium difficile cells and other bacterial cells susceptible to lysis upon contact with a polypeptide of SEQ ID NO: 1 (see Tables 1 and 2, below). Preferably, the microbial cells comprise or consist of Clostridium difficile cells. Thus, the polypeptides having the cell lysing activity of an endolysin from a bacteriophage of Clostridium difficile may be used to diagnose diseases and conditions associated with infection with Clostridium difficile cells (such as Clostridium difficile-associated disease, CDAD). Most preferably, the microbial cells comprise or consist of cells of Clostridium difficile ribotype 027.

In such diagnostic uses and methods, lysis of cells may be detected using methods well known in the art. For example, levels of ATP may be measured as an indicator of cell lysis.

Such diagnostic approaches are well established for endolysins from other systems, such as Listeria endolysins (for example, see Loessner et al., 2002, Mol Microbiol 44, 335-49; Kretzer et al., 2007, Applied Environ. Microbiol. 73:1992-2000, the disclosures of which are incorporated herein by reference; suitable assays are also available commercially, for example from Profos, Germany [see their website at www.profos.de/content/view/164/69/lang,en/]).

Exemplary embodiments of the invention are described in the following non-limiting examples, with reference to the following figures:

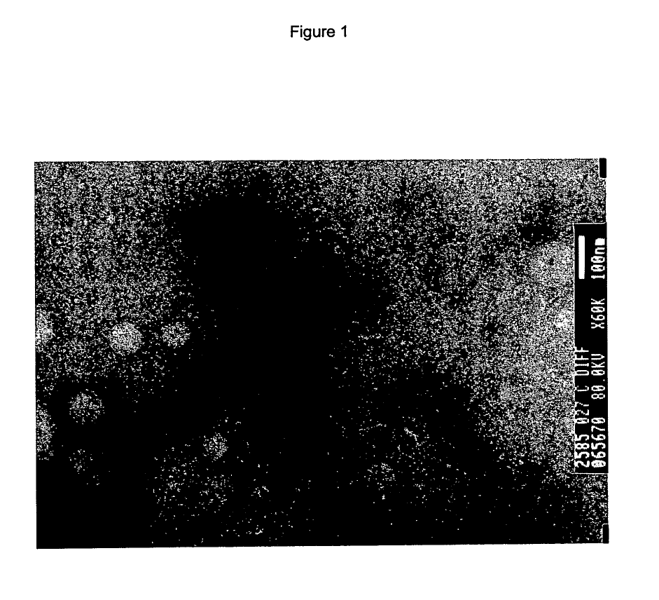

FIG. 1. Electron micrograph of φCD27. Samples were negative-stained in saturated uranyl acetate.

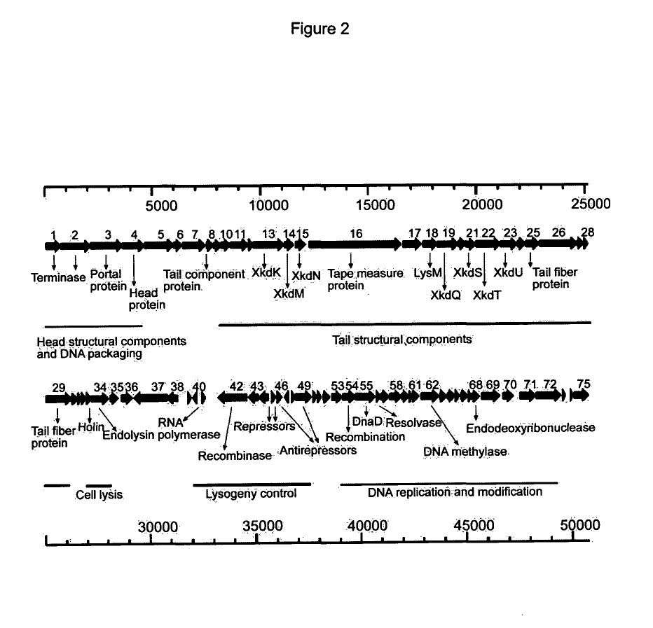

FIG. 2. φCD27 genome map showing predicted ORFs. Arrows indicate the directions of transcription. Proposed functional modules are marked based on BLAST results and similarity to published sequences of φCD119, φC2, and C. difficile strain 630 prophages.

FIG. 3. Nucleotide sequence of φCD27 lysin, SEQ ID NO 7.

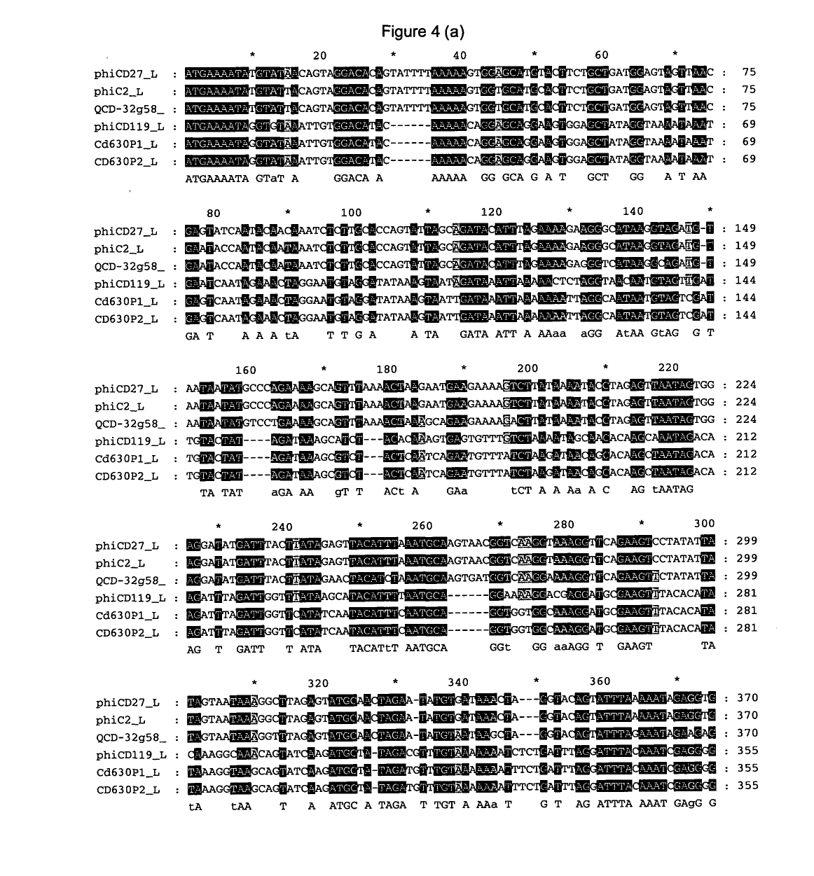

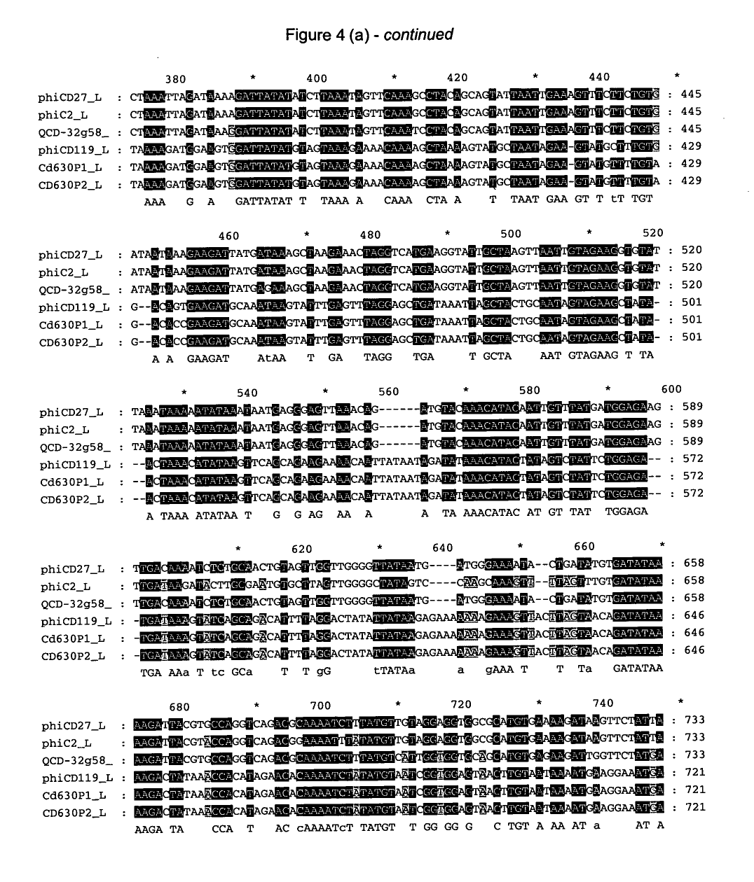

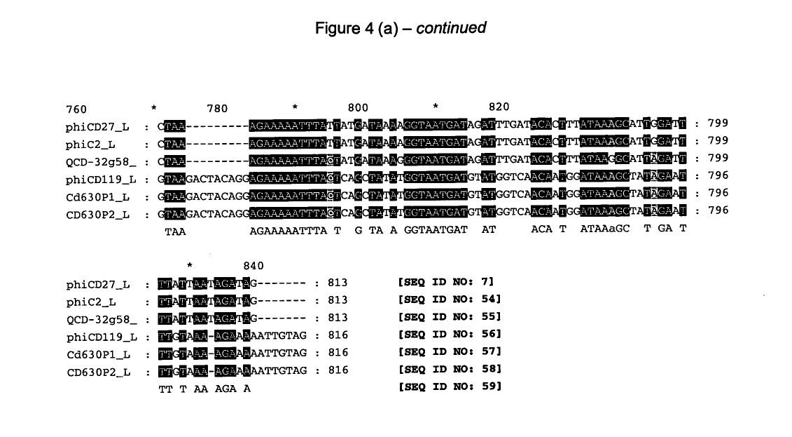

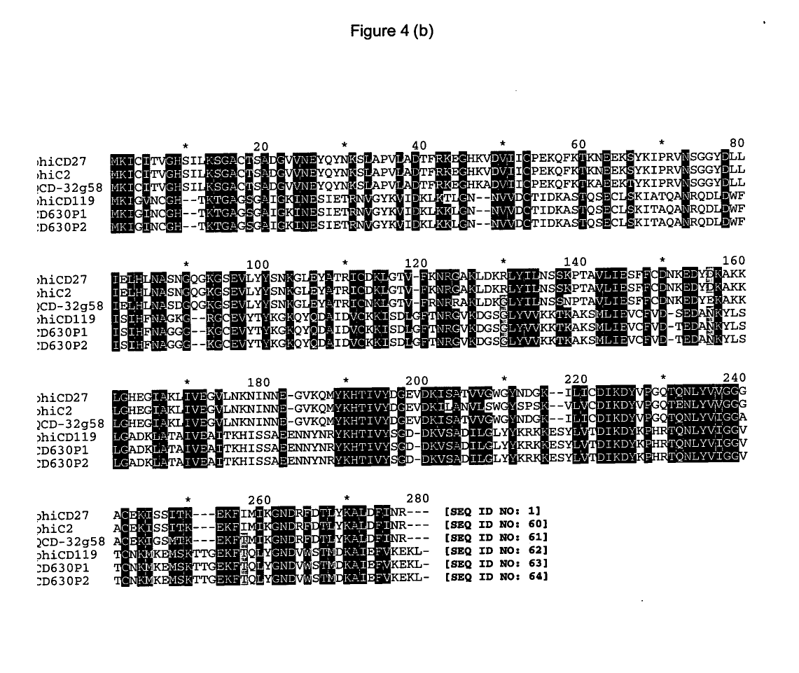

FIG. 4. Alignment of φCD27 (a) nucleotide and (b) inferred amino acid sequence with published C. difficile bacteriophage (φC2 (32); φCD119 (31)), or prophage (CD630 prophage 1 and 2 from sequenced genome (36)) sequences. Alignment performed with AlignX, Vector NTI Advance 10, Invitrogen. φCD27 amino acid sequence is SEQ. ID. 1

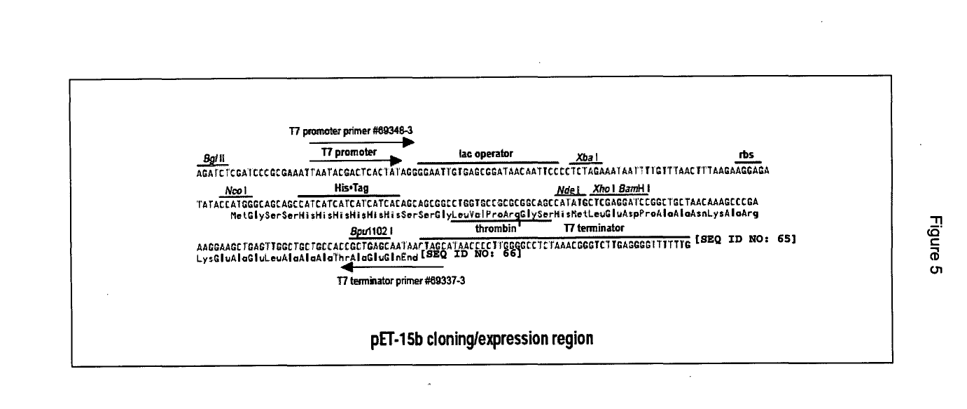

FIG. 5. Cloning site of pET15b vector (Novagen).

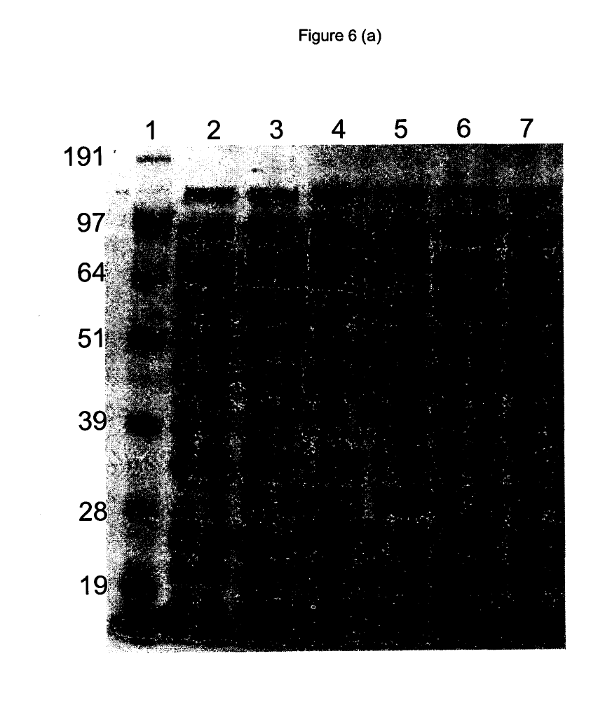

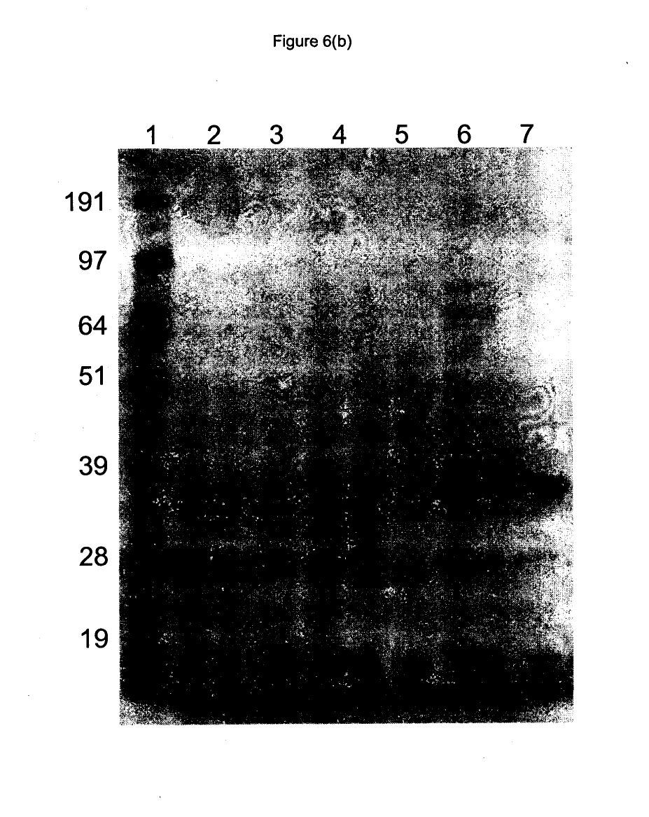

FIG. 6. (a) Gel analysis of crude protein lysates from E. coli expressing φCD27 lysin. Lane 1 SeeBlue marker (Invitrogen, sizes 191, 97, 64, 51, 39, 28 and 19 kDa), lanes 2-5 BL21(DE3)pET15bφCD27L total protein extracts. Lanes 2-4 extracts induced for 3 h with IPTG—2 and 3 extracted with 20 mM Tris-HCl pH 8, 50 mM NaCl, 3 including protease inhibitor (Roche Complete mini EDTA-free) and 4 extracted with denaturing buffer (8M urea, 0.1M NaH₂PO₄, 0.01M Tris-HCl pH 8.0). Lane 5 uninduced control extracted with 20 mM Tris-HCl pH 8, 50 mM NaCl. Lanes 6 and 7 BL21(DE3)pET15bCD630L1 total protein extracts extracted with 20 mM Tris-HCl pH 8, 50 mM NaCl, lane 6 only induced for 3 h with IPTG. (b) Western analysis of gel (a) with 6×His antibody.

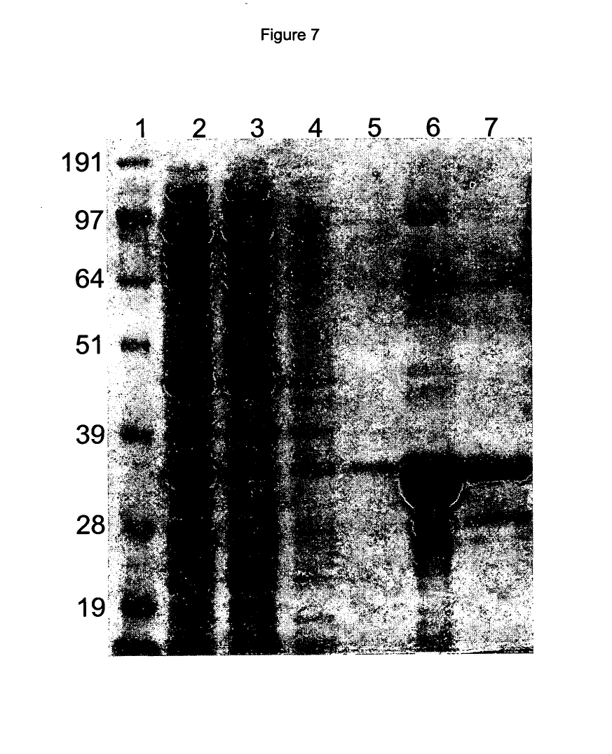

FIG. 7. Gel analysis of NiNTA column-purified His-tagged φCD27 lysin. Lane 1 SeeBlue marker (Invitrogen, sizes 191, 97, 64, 51, 39, 28 and 19 kDa), lanes 2-5 BL21(DE3)pET15bφCD27L total protein extracts after induction with IPTG. Lane 1 crude lysate, lane 2 column flow-through, lane 3 primary wash effluent, lane 4 secondary wash effluent, lane 5 primary eluate (E1, 1 ml), lane 6 secondary eluate (E2).

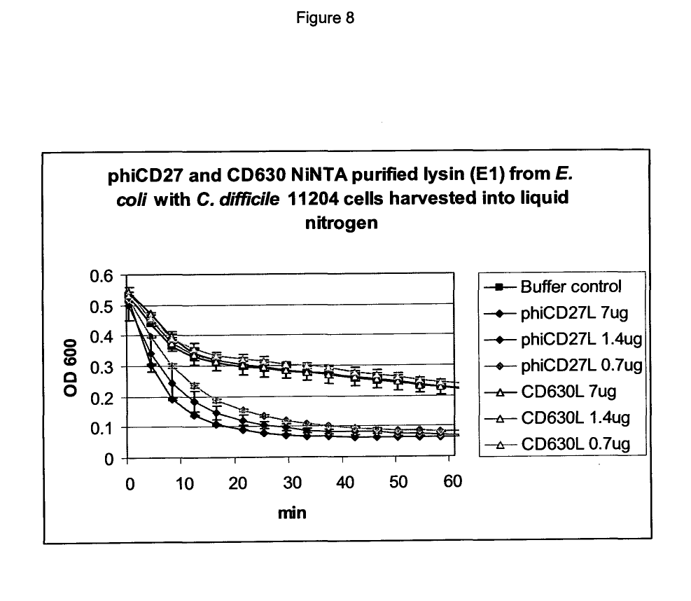

FIG. 8. Bioscreen lysis assay with cells of C. difficile 11204 grown to end log, flash frozen in liquid nitrogen then resuspended in PBS. φCD27 lysin and CD630 lysin were expressed in E. coli and purified using the His tag on a NiNTA column (see FIG. 6). 270 μl cells were added to 30 μl of dilutions of E1 extracts. Values are the means of duplicate assays+/−standard deviation. The cell lysis with the CD630L1 extract was equivalent to that seen in the buffer-only control.

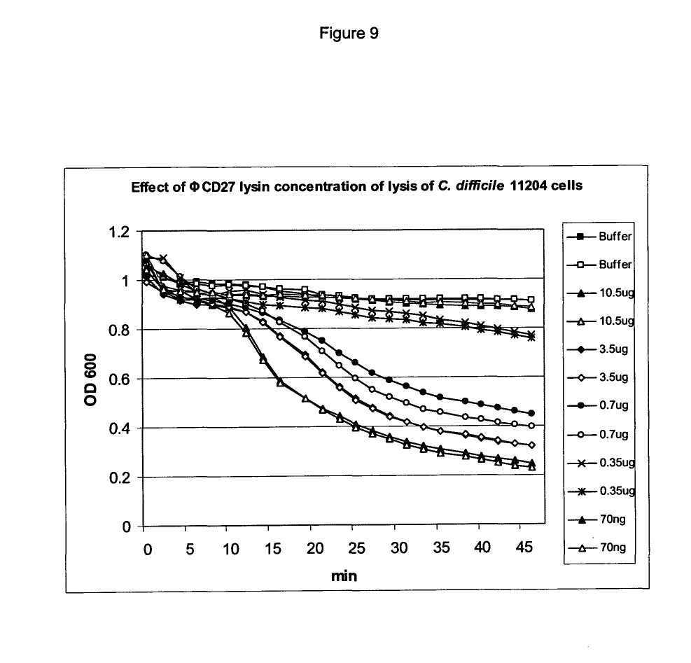

FIG. 9. Bioscreen lysis assay with cells of C. difficile 11204 grown to end log, harvested by centrifugation at 4° C. then resuspended in PBS to give an OD of between 1-1.5. φCD27 lysin was expressed in E. coli and purified using the His tag on a NiNTA column (see FIG. 6). 270 μl cells were added to 30 μl samples of eluate 1 (E1) diluted with elution buffer to give a range of concentrations from 10.5 μg to 0.35 ng per assay. The use of fresh cells gave significantly less lysis in the buffer-only control. No difference to buffer-only control was seen with less than 70 ng NiNTA-purified protein.

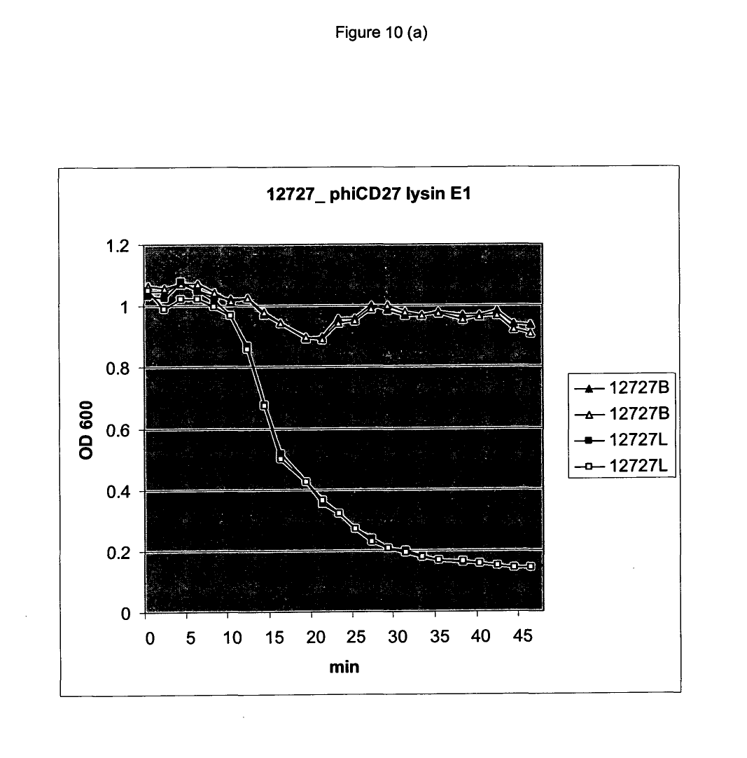

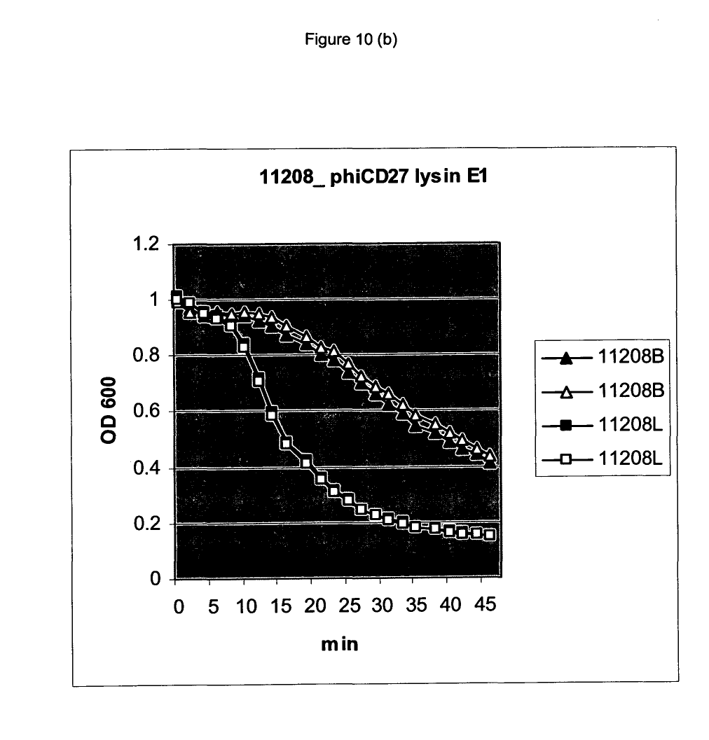

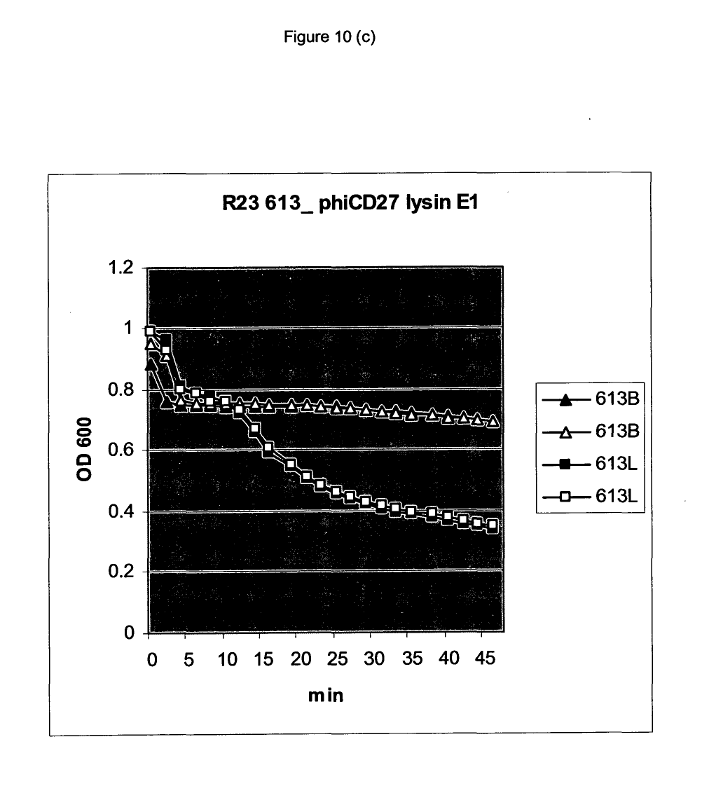

FIG. 10. Bioscreen lysis assays of φCD27 lysin added to C. difficile cells to test the spectrum of activity. Cells were incubated with 3.5 μg NiNTA-purified protein (E1) produced from E. coli. Of the 30 strains tested all were sensitive, including the host strain 12727 and bacteriophage φCD27-insensitive strains 11208 and hypervirulent ribotype 027 R23 613. Incubations were in duplicate with either buffer (B) or lysin (L).

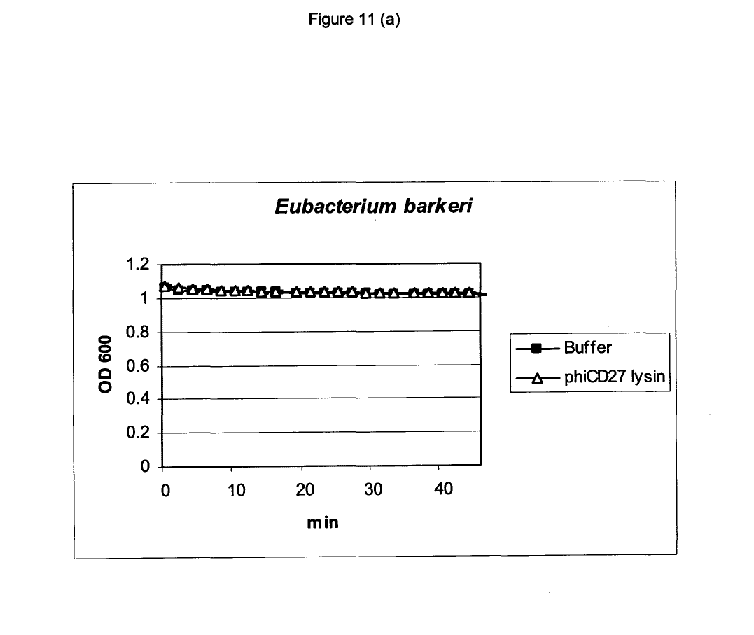

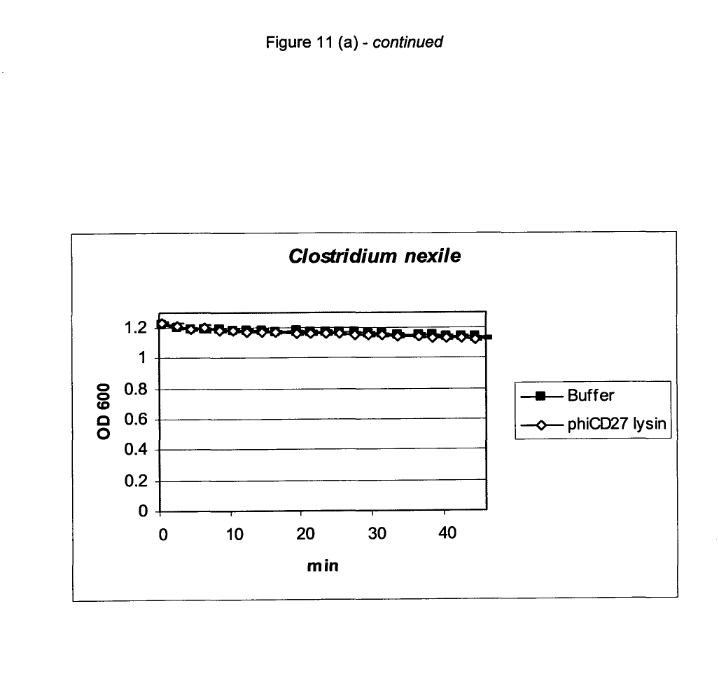

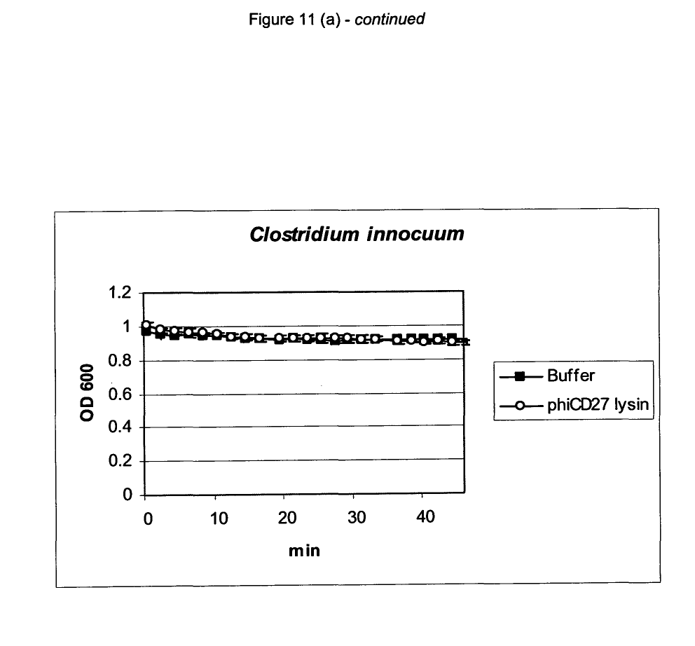

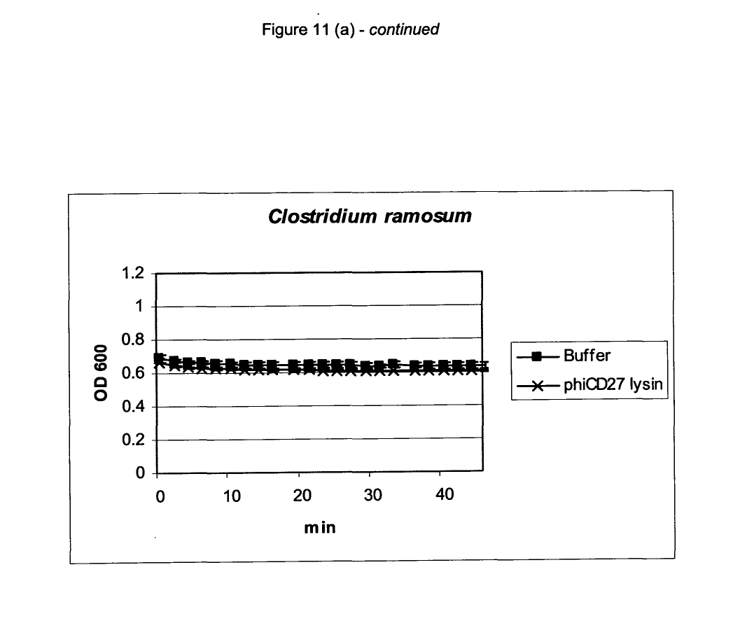

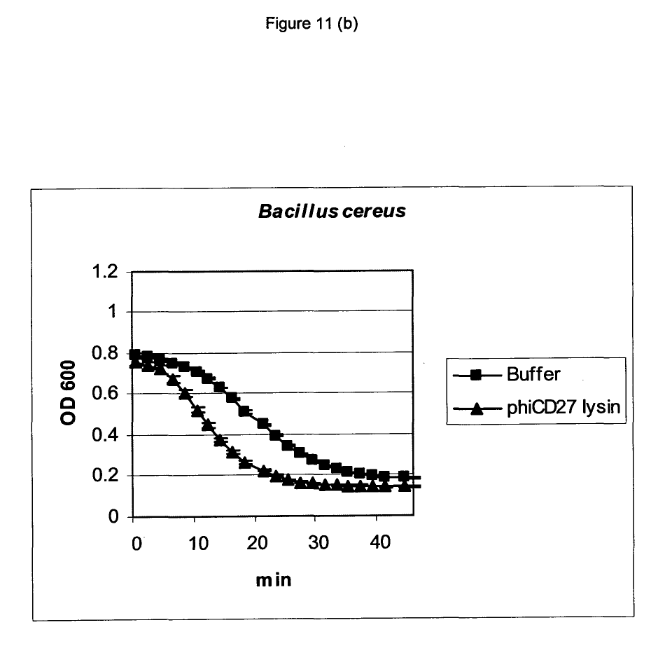

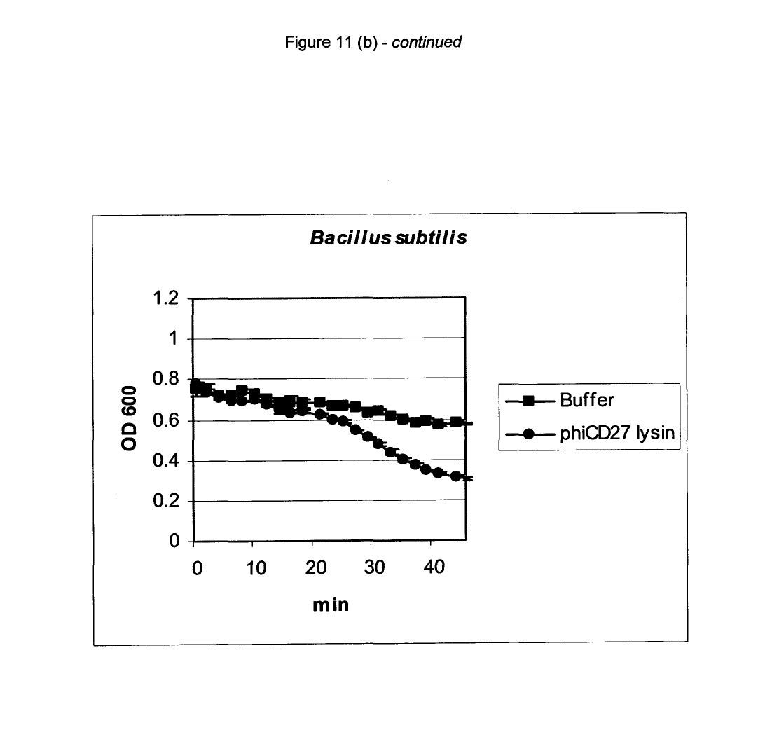

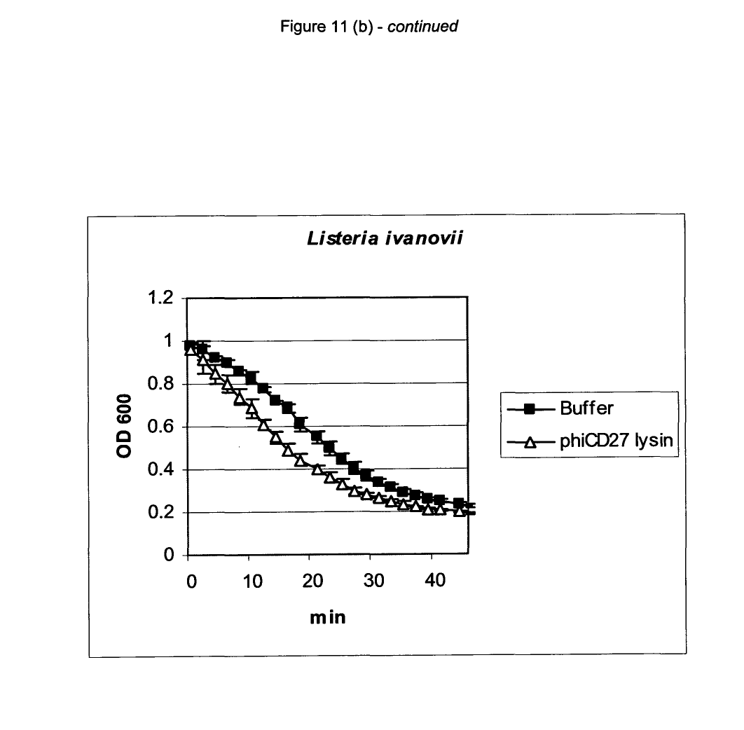

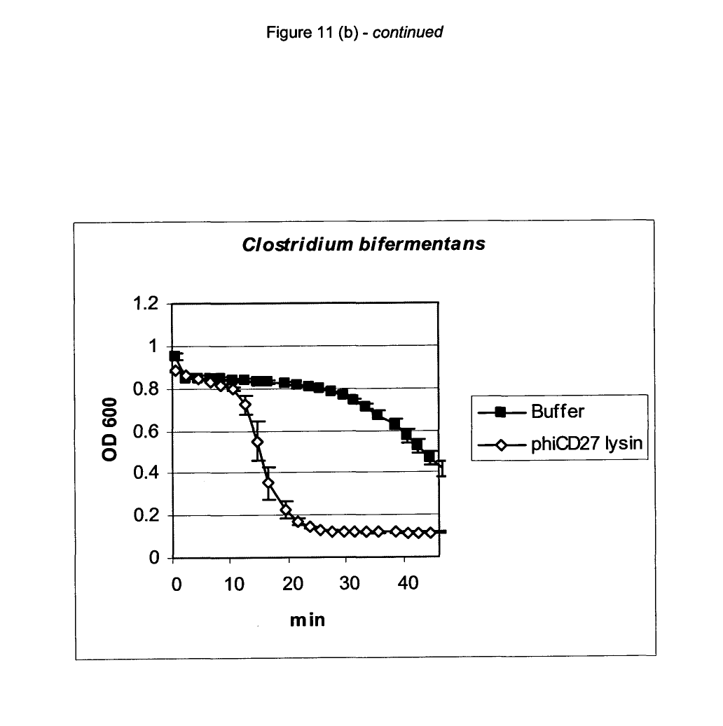

FIG. 11. Activity of φCD27 lysin against Clostridium species and prevalent gut bacteria. Cells were harvested at late stationary phase, resuspended in PBS then incubated with 7 μg NiNTA-purified protein (E1) produced from E. coli. Results are the mean of duplicate assays+/−standard deviation. The φCD27 lysin did not produce cell lysis in the majority of species (a, and see Table 2). Exceptions (b, and see Table 2) included a rapid lysis of Clostridium bifermentans and C. sordelli, lysis of Bacillus cereus and, with a longer lag phase, B. subtilis, and a slight effect on Listeria ivanovii (b).

FIG. 12. pH profile of φCD27 lysin activity. C. difficile 11204 cells were resuspended in PBS adjusted to a range of pHs and activity of the Ni-NTA-purified lysin E1 produced from E. coli was measured in the bioscreen as before.

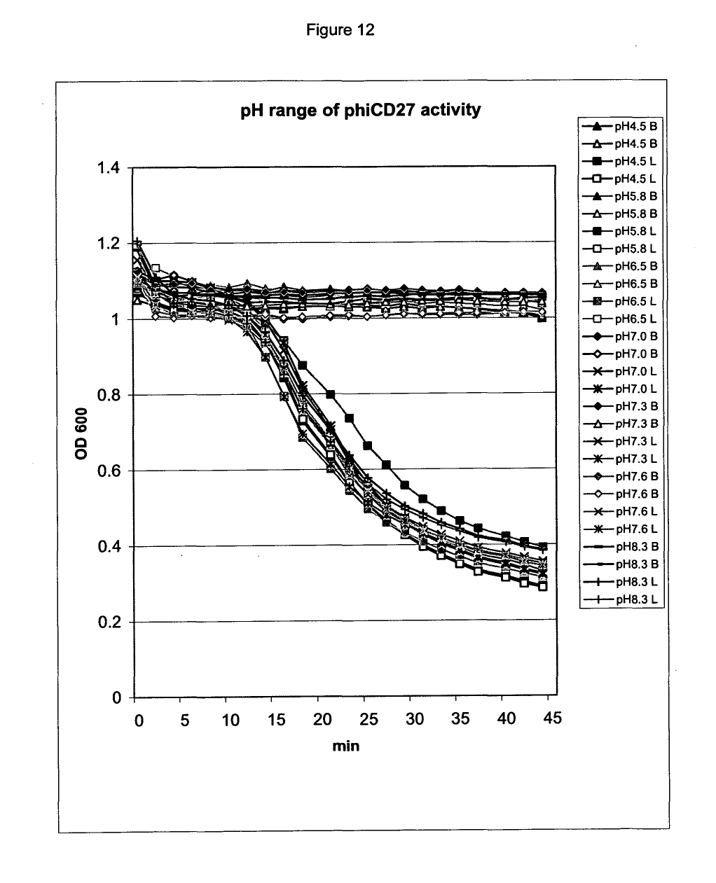

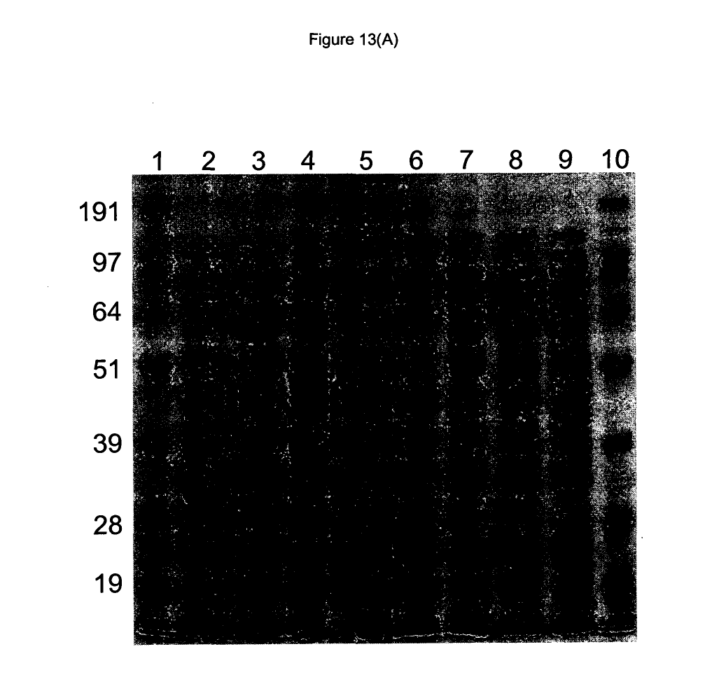

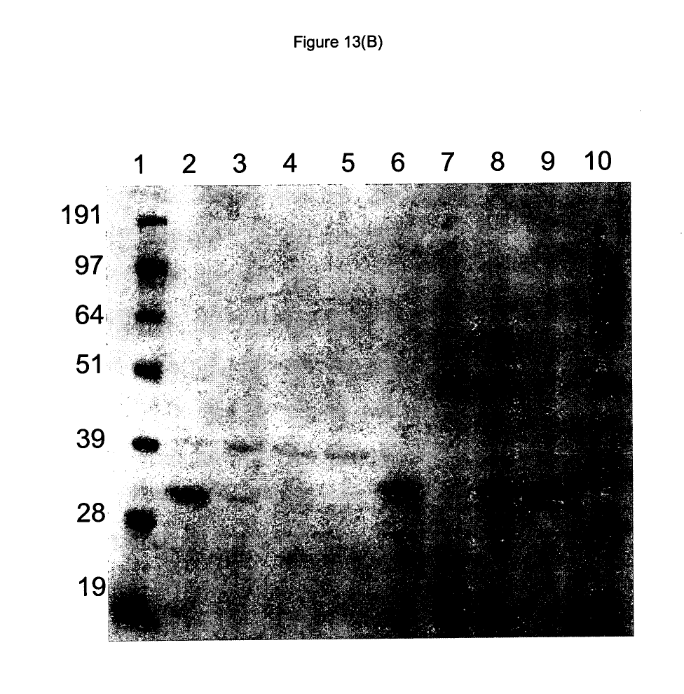

FIG. 13. (a) Gel analysis of crude protein lysates from Lactococcus lactis expressing φCD27 lysin. Lanes 1 and 10 SeeBlue marker (Invitrogen, sizes 191, 97, 64, 51, 39, 28 and 19 kDa), lanes 2-5 L. lactis UKLC10 containing phiCD27LpUK200HIS (2,3) or an empty vector pUK200HIS control (4,5), induced for 5 h (2,4) or uninduced (3,5). Lanes 6-9 E. coli BL21(DE3) containing phiCD27LpET15b (6, 8, 9) or the empty vector control (7) all induced for 4 h (10 μg per lane). All proteins were extracted in 20 mM Tris-HCl pH 8, 50 mM NaCl except lanes 8 (20 mM sodium phosphate pH 8) and 9 (50 mM Tris-HCl pH 7.5. (b) Western analysis of gel (a) with 6×His antibody.

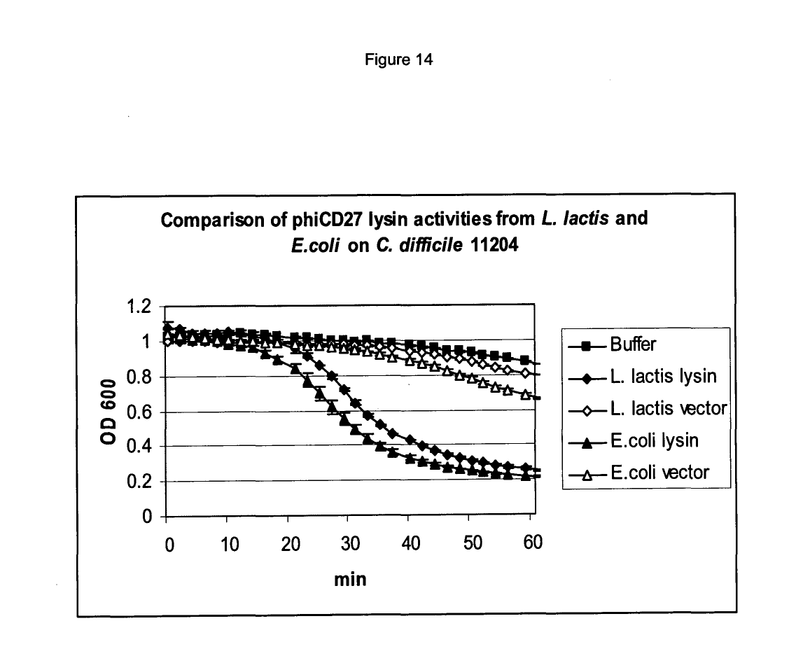

FIG. 14. Bioscreen assay of crude protein extracts from phiCD27 lysin-expressing E. coli and L. lactis incubated with fresh cells of C. difficile strain 11204 compared to extracts from empty vector controls. 50 μg protein was used in each assay, results are the mean of duplicate assays+/−standard deviation.

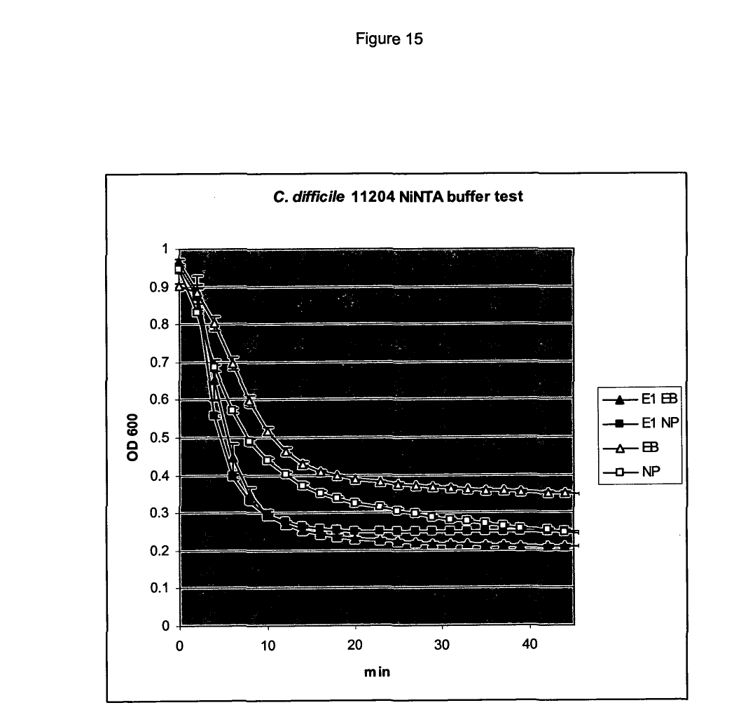

FIG. 15. Bioscreen assay of the Ni-NTA-purified lysin E1 produced from E. coli showing the activity of the original extract compared to that of an aliquot which had been through a zeba buffer exchange column (Pierce) into 20 mM sodium phosphate pH 6.0. Lysins and buffer controls were incubated with flash-frozen cells of C. difficile strain 11204 and results are the means of duplicate assays+/−standard deviation.

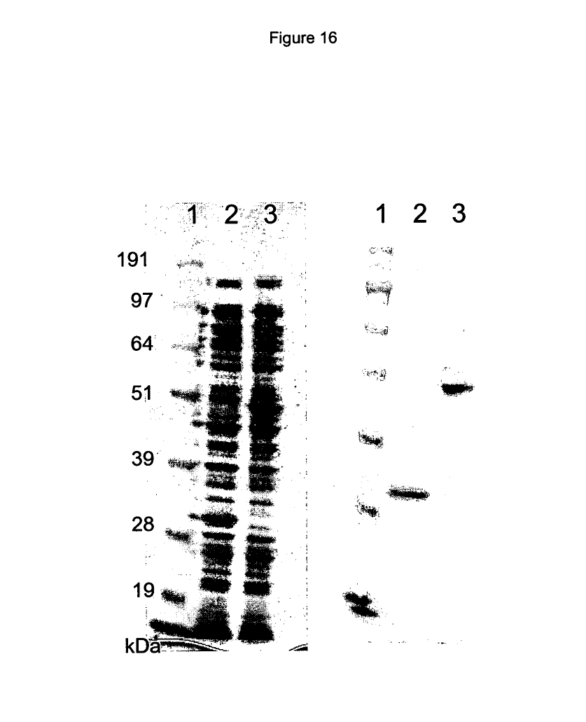

FIG. 16. SDS-PAGE of crude cell extracts of LM4-CD27L (lane 2) and LM4-CD27LE (lane 3) and the corresponding Western blot highlighting the His-tagged proteins. Proteins were extracted in 20 mM sodium phosphate pH 6.0 and 10 μg aliquots were electrophoresed on a 10% Bis-Tris NuPage gel in MOPS buffer (Invitrogen). Lane 1, SeeBlue marker.

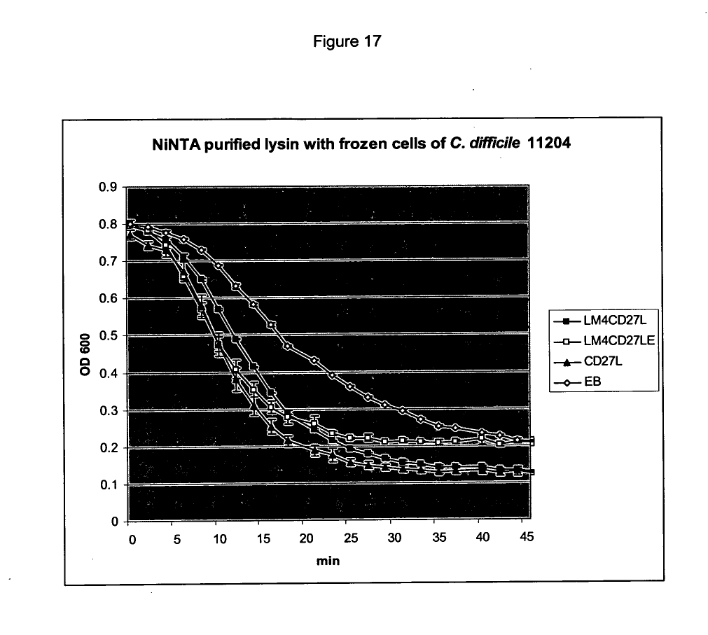

FIG. 17. Bioscreen analysis showing lysis of C. difficile strain 11204 cells grown to mid-log then flash frozen in liquid nitrogen. Cells were incubated with 10 μg NiNTA-purified E1 (eluate 1) from extracts of cells expressing LM4-CD27L, LM4-CD27LE the unaltered CD27L, or elution buffer as a control.

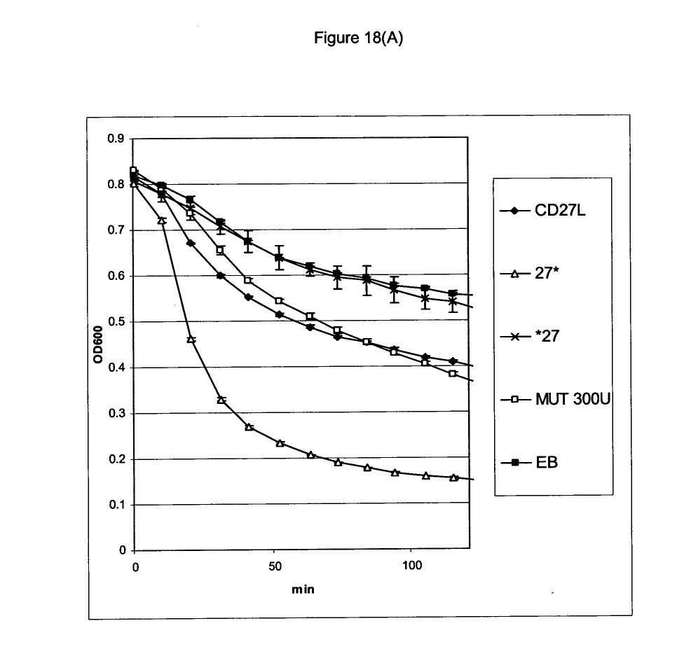

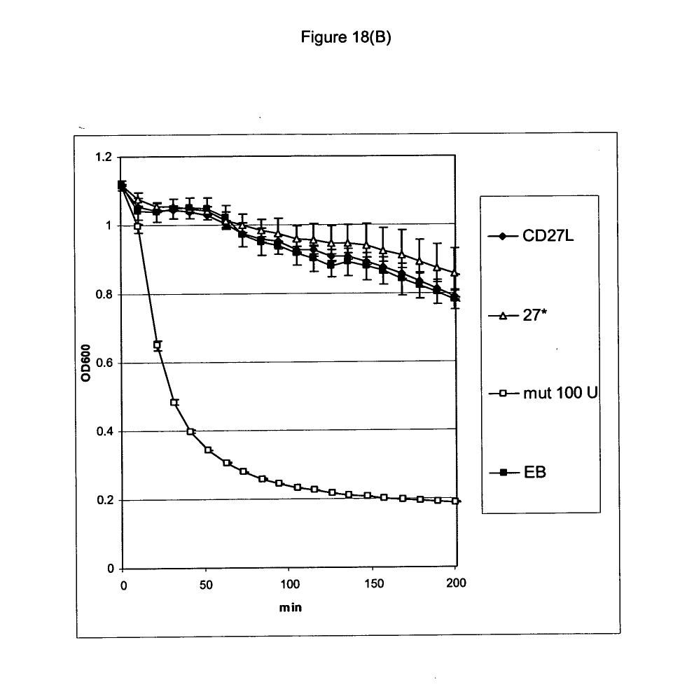

FIG. 18. Lysis assay of fresh cells incubated with 10 μg protein which had been partially purified using Ni-NTA spin columns (Qiagen). Extracts were from E. coli expressing the native endolysin CD27L [SEQ ID NO:1] (♦), the truncated endolysin “27*” [i.e. CD27L₁₋₁₇₉; amino acid residues 1 to 179 of SEQ ID NO:1] (Δ) or the C-terminal portion “*27” [i.e. CD27L₁₈₀₋₂₇₀; amino acid residues 180 to 270 of SEQ ID NO:1] (x), all extracted on the same day; controls were 100U mutanolysin (□) or elution buffer (▪). Extracts were incubated with a) C. difficile harvested at mid log; b) C. tyrobutyricum harvested at stationary phase. Results are the mean of duplicate assays+/−standard deviation.

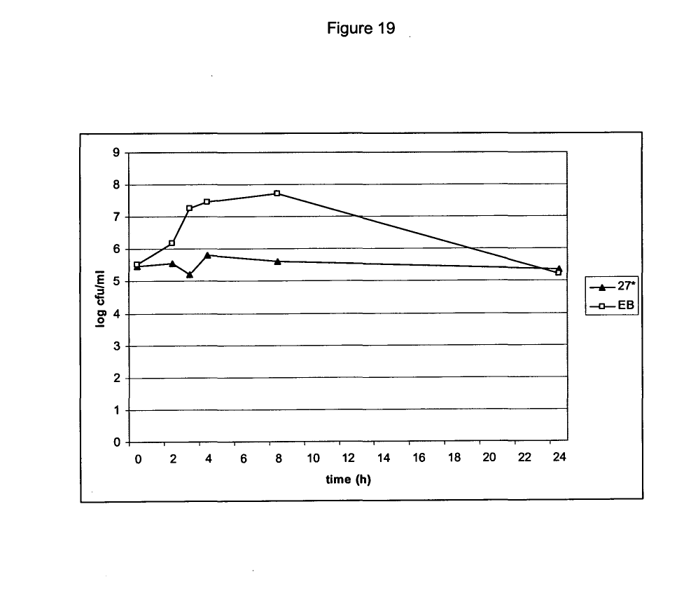

FIG. 19 C. difficile cell numbers after incubation of 1×10⁵ cfu in 3 ml BHI+C with either elution buffer (“EB”: □) or 1000 μg truncated endolysin CD27L₁₋₁₇₉ (“27*”; ▴).

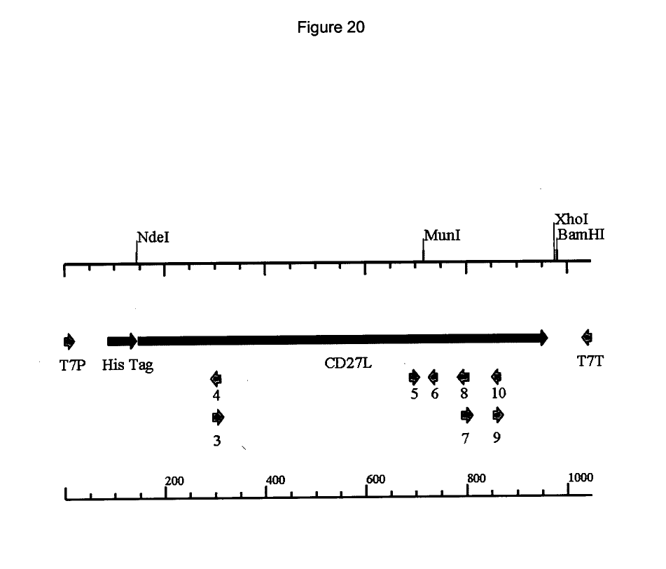

FIG. 20. Location of primers for production of cysteine mutants from CD27L-pET15b.

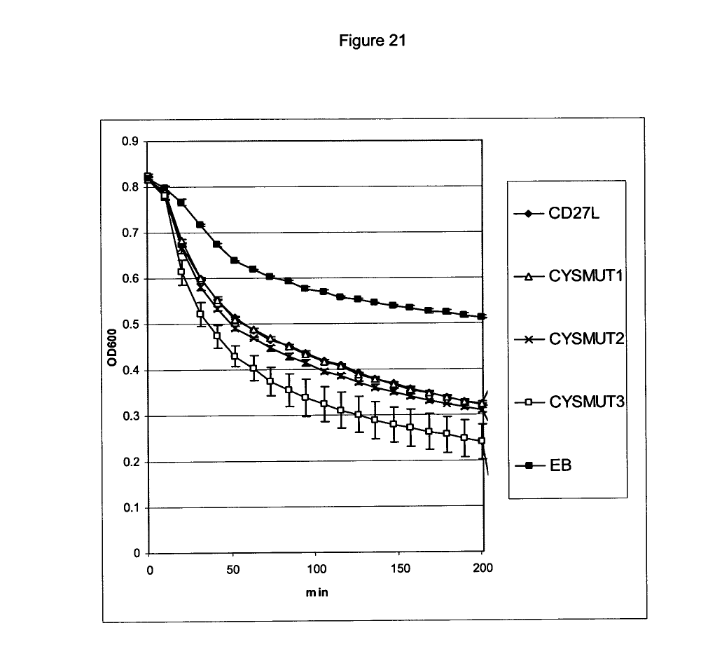

FIG. 21. Lysis assay of fresh C. difficile cells harvested at mid-log and incubated with 10 μg Ni-NTA spin column—purified protein from extracts of cells expressing cysteine mutants of CD27L CM1, CM2 or CM3 or the unaltered CD27L, EB—elution buffer negative control. Results are the mean of duplicate assays+/−standard deviation.

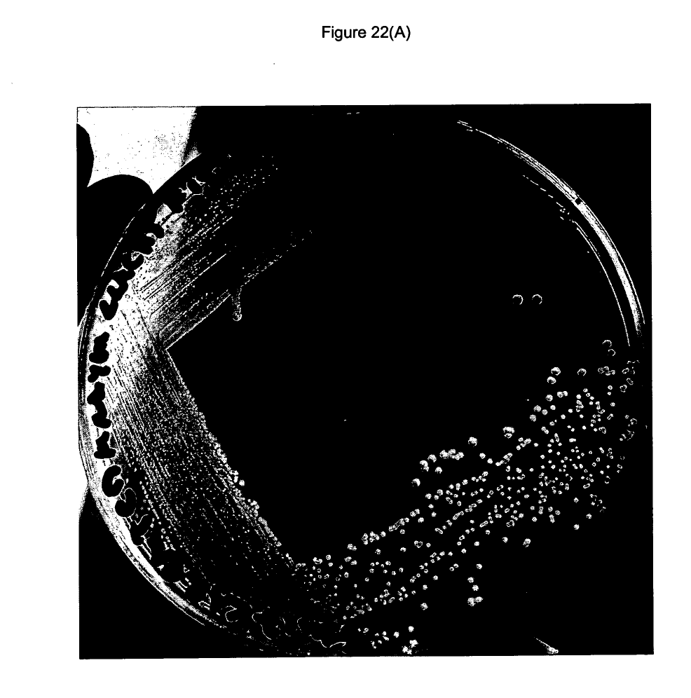

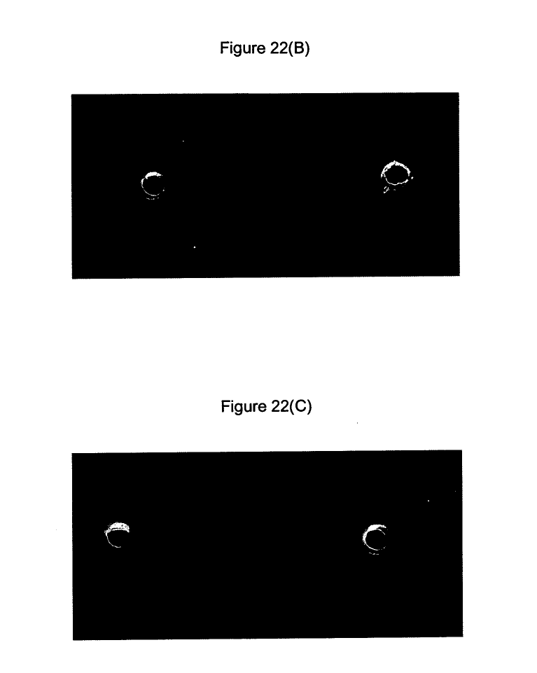

FIG. 22 a) Lysis of autoclaved C. difficile cells embedded in GM17 agar caused by external production of truncated endolysin CD27L1-179 (“27*”) from L. lactis FI5876-PnisASLPmodHis27*-pTG262. Plates were incubated for 3 d at 30° C.

b),c) lysis of autoclaved C. difficile cells embedded in PBS agar from concentrated supernatants and crude protein extracts of 6 h cultures of L. lactis FI5876-pTG262 (left) and FI5876-PnisASLPmodHis27*-pTG262 (right). b) 15 μl amicon-concentrated supernatants (concentration c. 20-fold) and c) 15 μl crude protein extract (equivalent to 22.5 μg total protein per well).

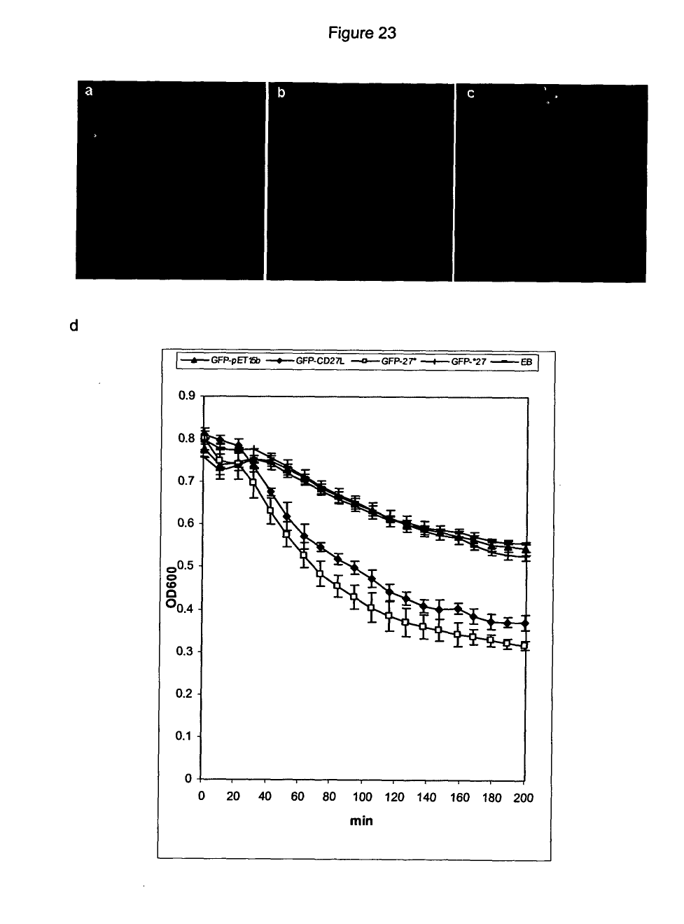

FIG. 23 Binding and lytic activity of GFP translational fusions with C. difficile. Binding assays contained 7.5 μM Ni-NTA purified GFP-CD27L (a), GFP-27* (b) and GFP-*27 (c); lysis assays (d) contained 10 μg protein incubated with fresh cells, values are the mean of duplicate assays+/−SD

### EXAMPLES

### Example A Characterisation of the Endolysin of SEQ ID NO:1

### Background

The exploitation of bacterial viruses as antimicrobial agents has experienced something of a renaissance in recent years. In part, this reflects the need to find alternatives to conventional antibiotics following the continued emergence of drug resistant pathogens. Recent reviews highlight this potential, but also emphasize limitations that are inherent in the use of bacteriophages (7, 8).

In general, bacteriophages exhibit significant strain specificity, meaning that they are only active against a restricted range of individual strains. The dynamics of the interaction between a bacteriophage and its bacterial host involve the ready selection of host mutants that are resistant to bacteriophage attack. Other issues of concern include the potential contamination of bacteriophage preparations with viable host bacteria and the potential for bacteriophages to contribute to gene flow and the spread of virulence factors (9). The carriage of toxin genes by bacteriophages is especially well documented, and examples include cholera toxin (10), botulinum toxin (9), shiga toxin (11) and diphtheria toxin (9). Despite these reservations, bacteriophages have been used experimentally to control E. coli (12), Staphylococcus aureus (13) and vancomycin resistant Enterococcus faecium (14) in mouse models. Bacteriophage therapy is being investigated for the control of Campylobacter (15) and E. coli (16) in chickens. With respect to clostridia, a study that targeted C. difficile in the hamster model has been reported (17). Further, the FDA has recently extended GRAS approval to a bacteriophage (LISTEX™, EBI Food Safety) for the control of Listeria in all food products (18).

In addition to the use of intact bacteriophages, there is the possibility of using bacteriophage endolysins as antimicrobial agents. The final stage of the bacteriophage life cycle involves the lysis of the bacterial host cell to release the pool of newly replicated intact bacteriophage particles. In general, this is achieved by a two stage process in which the carefully timed production of a membrane disruptive holin allows a cell wall degradative endolysin to access its peptidoglycan target. The endolysin enzyme is not secreted but released from the cell by the action of the holin and by its own capacity to degrade the cell wall. Once released, the endolysin can attack peptidoglycan from outside the cell, a phenomenon that has been observed from the time of early bacteriophage studies: it is referred to as ‘lysis from without’. The structure of most characterised bacteriophage endolysins is modular, with a catalytic domain and a distinct cell wall binding domain (CBD). The catalytic domain can vary and in most cases it is either an amidase or a muramidase. The CBD has a lectin-like ability to recognise sugar motifs on the bacterial cell surface, and the varied specificity involved gives the endolysins their characteristic targeting to a specific taxonomic group (19, 20).

Gasson et al. pioneered the exploitation of bacteriophage endolysins both as novel antimicrobial agents and as the basis of a novel detection technology using Listeria and Clostridium as model systems (21). Subsequently, the potential of endolysins as targeted antimicrobial agents has been widely recognised (22) with published examples that target Bacillus anthracis (23), Streptococcus pneumoniae (24) and Enterococcus faecalis (25). With respect to Listeria, significant additional work has been undertaken by Martin Loessner at ETH, Switzerland (19, 20). In addition, an endolysin active against Clostridium perfringens has been characterised (26).

### Characterization of a Novel Bacteriophage Lysin and Methods of Use Thereof.

The temperate bacteriophage φCD27 was isolated from Clostridium difficile culture collection strain NCTC 12727. φCD27 was tested against 25 other C. difficile strains and shown to be effective against 4 other strains, including the type strain 11204. The bacteriophage genomic DNA was extracted and sequenced and the endolysin sequence identified by BLAST search. The sequence shows clear amino acid and nucleotide homology to published C. difficile bacteriophage endolysins (φCD119, φC2, prophages 1 and 2 in sequenced C. difficile CD630). The lysin was subcloned into pET15b and expressed in E. coli with a 6×His tag. The lysin was partially purified on a nickel column and shown to lyse both phage-sensitive and -insensitive strains, evidenced by a drop in optical density upon incubation at 37° C. Of 30 strains tested all showed lysis, including strains of the virulent ribotype 027. A number of other bacteria from a range of genera showed no susceptibility to the lysin. However some activity was observed against C. bifermentans, C. sordelli, Bacillus cereus, B. subtilis and very limited activity against Listeria ivanovii. Specific activity of the partially purified lysin varied depending on the C. difficile strain. Accordingly, the lysin disclosed herein represents a potent new weapon for the treatment and detection of C. difficile pathogenesis.

The lysin identified and characterized herein is a novel composition of matter which may be utilized to treat C. difficile infections and other bacterial infections in humans and in animals. According to this invention, the φCD27 lysin may be produced according to methods known in the art. It may be isolated for use from the virus grown for this purpose. Preferably, however, it is produced by recombinant means disclosed herein and by alternate means known to those skilled in the art. Relevant sub-portions of the molecule are characterized for their ability to specifically bind to bacteria and to lyse those bacteria. These molecular sub-portions may be produced and used separately or together as in the native molecule.

### Discovery, Cloning and Activity of φCD27 Lysin

Lysate production and activity assays were performed as described (27). C. difficile strain NCTC 12727 (available from the Health Protection Agency, Colindale, London—is deposited by S. Tabaqchali, St. Bart's Hospital, London in 1992 isolated from faeces) was grown for 24 h anaerobically at 37° C. in BHI+C (brain heart infusion medium, BHI (Oxoid), supplemented with vitamin K (10 μl 0.5% v/v/l) hemin (5 mg/l), resazurin (1 mg/l) and L-cysteine (0.5 g/l)). Bacteriophage production was induced for 24 h with mitomycin C (Sigma), at a final concentration of 3 μg/ml. Cultures were centrifuged at 4,000×g for 20 mins at 4° C. and supernatants were filtered through 0.45 μm filter units (Millipore) and stored at 4° C. The supernatant was spotted in 25 ul portions onto BHI plates (1.5% agar) overlaid with BHI soft agar (0.75%) incorporating 150 ul of an overnight C. difficile BHI+C culture, and incubated overnight anaerobically at 37° C. Cultures (see Table 1) were tested in duplicate and clear plaque formation from 12727 supernatant was identified on 4 strains—C. difficile 11204 (type strain), 11205, 11207 and 11209. Plaques from strain 11204 were picked with a sterile Pasteur pipette into 250 μl BHI+C and incubated overnight at 4° C. The presence of a bacteriophage—φCD27—was confirmed by electron microscopy, which indicated it belonged to the order Caudovirales (28)(FIG. 1). In total 25 strains of C. difficile were induced with mitomycin C and the supernatants cross-tested against all 25 strains. φCD27 was the only plaque-forming unit discovered by this method. The infrequency of bacteriophage discovery from C. difficile has also been noted in previous publications which found 2 bacteriophage producers from 94 isolates (29) or 3 producers from 56 isolates (30).

To increase the titre, 100 μl of the plaque eluate was mixed with 100 μl of a 24 h culture of C. difficile strain 11204 in 5 ml BHI soft agar and plated onto BHI agar. Overnight anaerobic incubation at 37° C. gave near-confluent lysis and elution for 2 h into 5 ml BHI+C gave a titre of 2×10⁶ pfu/ml. The titre was increased by consecutive incubations in 11204 liquid culture, growing the cells in 25 ml BHI+C cultures to early to mid-log phase, giving an optical density (OD) to allow a ratio of bacteriophage:cells of at least 4:1. This method gave complete clearing of the bacterial suspension and 2 passages gave a titre of 2.5×10¹¹ pfu/ml. For DNA extraction, cells at OD 0.3 were inoculated with filtered lysate to a multiplicity of infection of c. 7. An incubation of 3 h gave complete lysis and the supernatant was harvested and filtered as before and two 50 ml portions were used in a Qiagen λ midikit (Qiagen), giving a yield of c.160 μg bacteriophage genomic DNA.

Sequencing and assembly of the bacteriophage φCD27 genome was performed by the Biochemistry DNA Sequencing Facility (University of Cambridge, UK) using the Phred-Phrap program. The circular genome is 50,930 by and contains 75 proposed open is reading frames (orfs) (FIG. 2). Many of these show significant homology to identified bacteriophage ORFs, including those from C. difficile bacteriophages φCD119 (31) and φC2 (32). ORFs were analysed by Artemis (33) with BlastP searches (34, 35) which were run via BITS (Harpenden). The proposed φCD27 lysin sequence is 816 bp, coding for a 270 amino acid predicted protein which shows homology to N-acetylmuramoyl-L-alanine amidase. Both the nucleotide and amino acid sequences (FIG. 3) align to published sequences from C. difficile bacteriophages and prophages (FIG. 4), with the greatest homology (95.9% nucleotide and amino acid identity) being to φC2.

The φCD27 lysin sequence was amplified from genomic DNA using primers to create an NdeI site (CATATG) around the initial Met residue (primer CD27L_NDE, 5′-TTA CAT ATG AAA ATA TGT ATA ACA GTA GG [SEQ ID NO: 8], Sigma Genosys) and a XhoI site (CTCGAG) downstream of the coding sequence (primer CD27L_XHO, 5′-CAA CCA CCT CGA GTT GAT AAC [SEQ ID NO: 9], to facilitate subcloning in the expression vector pET15b (Novagen). Amplification was performed with high fidelity Phusion DNA polymerase (0.02 U/μl, Finnzymes) in a 50 μl reaction containing 1× Phusion buffer, 200 μM dNTPs, 0.5 μM of each primer, 200 ng genomic DNA template. Amplification conditions were an initial denaturation of 98° C. for 30 s followed by 30 cycles of denaturation (98° C. 10 s), annealing (58° C. 30 s) and extension (72° C. 15 s) then a final extension of 72° C. for 5 min. Blunt end PCR products were purified using SureClean (Bioline) and given 3′ A-overhangs in a 50 μl reaction containing 1× AmpliTaq buffer, 0.2 mM dATP and 1U AmpliTaq DNA polymerase (Applied Biosystems) incubated for 20 min at 72° C. Products were purified with SureClean then ligated into pCR2.1 using the TA cloning kit (Invitrogen). Ligation products were transformed into TOP10 chemically competent E. coli (Invitrogen) and positives were selected on L agar supplemented with 100 μg/ml ampicillin and overlaid with 40 μl of a 40 mg/ml X-gal solution for blue-white selection. Plasmid DNA was extracted using a plasmid mini kit (Qiagen) and inserts were sequenced using vector primers and the BigDye v3.1 sequencing kit (Applied Biosystems). A clone showing 100% sequence homology to the original lysin sequence but with the added NdeI and XhoI sites was restricted to release the insert. This was gel purified (Qiaex II, Qiagen) and ligated using Fast-Link DNA ligase (Epicentre), into pET15b so that the lysin sequence was expressed downstream of a 6-histidine tag under the control of the high expression T7 promoter with the IPTG-inducible lac operator (FIG. 5). Ligation products were transformed first into TOP10 cells for sequence confirmation then into chemically competent BL-21(DE3) cells (Invitrogen) for protein expression. The lysin sequence from prophage 1 of the sequenced C. difficile (36) was synthesised by Genscript Corp. (Piscataway, USA) into the vector pUC57 and subcloned for His-tagged expression in the same way using primers CD630L1_NDE (5′-TGC TCA TAT GAA AAT AGG TAT AAA TTG) [SEQ ID NO: 10] and M13 forward (5′-GTA AAA CGA CGG CCA GT) [SEQ ID NO: 11] which amplified the lysin with some vector DNA including a XhoI site.

His-tagged lysin was expressed as suggested by the manufacturer in BL-21(DE3) cells grown in 10 ml L broth with 100 μg/ml ampicillin to OD₆₀₀ 0.4 then induced with 0.5 mM IPTG (Melford Biosciences) for 3-4 h. Cells were harvested by centrifugation at 4000×g and 4° C. for 20 min then resuspended in 1 ml buffer (20 mM Tris-HCl pH 8, 50 mM NaCl) and transferred to 2 ml screw cap tubes. Crude protein lysate was obtained by cell disruption with 0.1 mm acid-washed glass beads (Sigma) in a FastPrep FP120 cell disrupter (Savant) with 4×30 s bursts (speed 10), incubating on ice for 5-10 min between bursts. Debris was pelleted by centrifugation at 13,000×g for 20 min at 4° C. and the supernatant stored at 4° C. Crude lysates were also produced from cells containing the lysin grown without IPTG induction and cells containing the empty pET15b vector grown with and without induction. Protein content was measured using the Bradford reagent (Bio Rad) and 10 μg portions were electrophoresed on 10% NuPage Novex Bis Tris gels in MOPS buffer (Invitrogen). Presence of the His-tagged lysin was confirmed by Western blotting using an anti His-Tag monoclonal antibody (Novagen). Proteins were transferred to PVDF membrane using NuPage buffer (Invitrogen) and detection was as described by Qiagen (Qiaexpress detection and assay handbook) with anti-mouse IgG as the secondary antibody and colorimetric detection with Sigma Fast BCIP/NBT alkaline phosphatase substrate. This demonstrated high expression of a His tagged band of c. 33 kDa in IPTG-induced lysates and also lower expression in uninduced lysates (FIG. 6).

Lysis of C. difficile cells of strains 11204 and 11207 by crude lysates was assessed using the method described by Loessner et al (37). Cells of strain 11204 were grown to end-log phase, 1.8 ml aliquots were harvested by centrifugation into screw cap tubes (13,000×g, 2 min) and pellets were flash-frozen in liquid nitrogen and stored at −20° C. Pellets were resuspended on ice in 900 μl 20 mM Tris-HCl pH 8 and added to a cuvette containing 100 μl crude protein lysate then the drop in OD₆₀₀ was monitored for 1 h with mixing before reading. With this system the C. difficile cells showed a certain amount of lysis in the buffer, although lysis with the φCD27 lysin crude extract was more rapid and extensive. However, a subsequent test with the induced empty pET15b vector crude lysate demonstrated an equivalent lysis, suggesting the activity of E. coli lysozymes. To avoid this problem the φCD27 and CD630L1 lysins were affinity-purified using the Qiagen NiNTA kit. BL-21(DE3) cells were grown to OD₆₀₀ 0.6 in 250 ml L broth containing 100 μg/ml ampicillin then induced for 5 h with IPTG at a final concentration of 1 mM. Cells were harvested by centrifugation at 4000×g and 4° C. for 20 min and pellets stored at −20° C. Protein was purified under native conditions and purification was confirmed by NuPage gel analysis (FIG. 7). This method produced partially purified protein of which the majority was lysin, with a yield of 2.3 mg total protein in the first φCD27 eluate (E1) and 0.5 mg in the second (E2). Incubation of dilutions of the E1 eluate showed rapid lysis of strain 11204 cells compared to an eluate from cells prepared in the same way but expressing the empty pET15b vector However, CD630L1 E1 eluate did not lyse strain 11204 and there was no synergistic effect with φCD27 lysin.

Lysis assays continued in multiwell plates using the Bioscreen C (Labsystems) and NiNTA-partially purified lysin extract in elution buffer (EB, Qiagen). Initially assays used c.7 μg protein in a total volume of 30 μl EB and 270 μl cells as in the spectrophotometer assays. Assays were set up on ice then transferred to the Bioscreen C pre-heated to 37° C. and the program was run as follows—sampling every 2 min with 10 s shake before sampling at an optical density of 600 nm. Each assay was run with two wells of buffer only and 2 wells of lysin, all 4 wells being inoculated from the same bacterial cell suspension. In this system lysis in the lysin wells of sensitive strains was rapid—a difference being notable within 5 min. However, lysis of the cells in buffer-only controls was also obvious, albeit at a much slower rate than the lysin-induced lysis (FIG. 8).

When both C. difficile and other bacterial cells were grown to end log and harvested onto ice without freezing then assayed as soon as possible the buffer-only lysis was reduced or totally absent (FIG. 9) and lysis of all other species was absent with the notable exceptions of Clostridium bifermentans, Clostridium sordelli, Bacillus cereus and to lesser extents B. subtilis and Listeria ivanovii (FIG. 11, Table 2). Additional strains representative of the AT rich Clostridium-like component of the GI tract microflora were tested for sensitivity to the φCD27 lysin. As shown in Table 3, none of those tested were sensitive to the lysin.

Using fresh cells gave a less rapid onset of lysis in C. difficile with a notable lag of up to 12 mins (FIG. 9). All C. difficile strains were re-tested using 3.5 μg lysin isolated from a second NiNTA column (tested to show equal lysis to the first purification; FIG. 10). In both cases, using fresh or frozen cells, the sensitivity profiles were the same with all 30 strains showing clear sensitivity to the lysin (Table 1).

The pH profile of the φCD27 lysin was tested using the sensitive strain 11204—activity showed very little variation within a fairly large pH range, tested at pH 4.5, 5.8, 6.5, 7.0, 7.3 (usual pH of PBS), 7.6 and 8.3 (FIG. 12). A dilution series showed that although the activity with 10.5 μg protein in the 300 μl assay was maximal, good lysis was also seen with 3.5 μg and 0.7 μg. However, 0.35 μg gave a response only slightly below the buffer controls and lower amounts showed no lysis within the 45 min assay (FIG. 9).

The delivery of the φCD27 lysin to the GI tract could be achieved by the use of physical encapsulation or a recombinant commensal microorganism such as a member of the lactic acid bacteria. Lactococcus lactis has established potential in this regard and thus sub-cloning and expression of the φCD27 lysin in this species was demonstrated. The φCD27 lysin sequence was subcloned into the vector pUK200His. This is a derivative of the nisA translational fusion plasmid pUK200 (38) constructed by restriction of pUK200 with NcoI, end-filling, then insertion of an oligomer encoding a 6-histidine tag (AGT CAT CAC CAT CAC CAT CAC GC) [SEQ ID NO: 12] downstream from the nisin-inducible promoter. When recircularised, this recreated an NcoI site for subcloning (Horn et al., unpublished). Vector pUK200His was restricted with NcoI and end-filled with T4 DNA polymerase (Promega) to create the first ATG codon for a translational fusion under control of the nisA promoter. The phicd27l sequence was amplified from the CD27L-NDE . . . CD27L-XHO PCR product subcloned in pCR2.1 (see above). Primers CD27LCOD2_F (5′-AAA ATA TGT ATA ACA GTA GGA CAC) [SEQ ID NO: 13] and M13 forward (5′-GTA AAA CGA CGG CCA GT) [SEQ ID NO: 14] amplified the full sequence from the second codon AAA and some of the vector sequence, giving an EcoRI site immediately after the lysin coding sequence. Amplification was as described above but with an annealing temperature of 56° C. Both the PCR product and the NcoI-cut, end-filled pUK200His vector were restricted with EcoRI and ligated together to create the His-tagged translational fusion under control of the nisA promoter. Ligation products were transformed into electrocompetent E. coli strain MC1022 for sequence verification, with positive transformants being selected on chloramphenicol (15 μg/ml). Purified plasmid preparations were then transformed into electrocompetent Lactococcus lactis strain FI10676 and selected on GM17 agar supplemented with 5 μg/ml chloramphenicol.

L. lactis strains expressing pUK200His-phiCD27L or pUK200His empty vector control were grown in 10 ml GM17 broth with 5 μg/ml chloramphenicol at 30° C. static. 100 μl of an overnight culture was used to inoculate pre-warmed broth and the culture grown to midlog (OD₆₀₀ 0.5). Expression was induced with 1 ng/ml nisin for 5 h at 30° C. and crude protein lysates were produced as described for E. coli in 20 mM Tris-HCl pH 8.0, 50 mM NaCl. A demonstration of lactococcal expression of φCD27 lysin is presented as a protein gel analysis (FIG. 13). The sensitivity of Clostridium difficile strain 11204 to the φCD27 endolysin expressed in Lactococcus lactis was demonstrated using crude protein extracts as is shown in FIG. 14.

TABLE 1
(overleaf) Strains of Clostridium difficile used in bacteriophage
and lysin assay tests.

| Strain | Source | Details | Bacteriophage | Lysin |
|---|---|---|---|---|
| NCTC 11204 | a | Meconium from | S | L |
| N1 | | neonates, 1970 | | |
| NCTC 11205 | a | Meconium from | S | L |
| N2 | | neonates, 1970 | | |
| NCTC 11206 | a | Meconium from | R | L |
| N3 | | neonates, 1970 | | |
| NCTC 11207 | a | | S | L |
| N4 | | | | |
| NCTC 11208 | a | | R | L |
| N5 | | | | |
| NCTC 11209 | a | | S | L |
| N6 | | | | |
| NCTC 11223 | a | Faeces | R | L |
| 335722 | | | | |
| NCTC 12726 | a | faeces, 35S methionine | R | L |
| | | protein type A | | |
| NCTC 12727 | a | faeces, 35S methionine | R | L |
| | | protein type B | | |
| NCTC 12728 | a | faeces, 35S methionine | R | L |
| | | protein type C | | |
| NCTC 12729 | a | faeces, 35S methionine | R | L |
| | | protein type D | | |
| NCTC 12731 | a | faeces, 35S methionine | R | L |
| | | proteint ype Y | | |
| NCTC 13287 | a | R7404 | nt | L |
| NCTC 13307 | a | Strain 630 | nt | L |
| NCTC 12731 | a | faeces, 35S methionine | R | L |
| | | protein type W | | |
| NCTC 13366 | a | R2029 | nt | L |
| NCTC 11382 | a | Blood culture, | R | L |
| 74/1451 | | New Zealand, 1980 | | |
| R23 521 | b | Ribotype 118 | R | L |
| R23 524 | b | Ribotype 001 | R | L |
| R23 613 | b | Ribotype 027 | R | L |
| R23 614 | b | Ribotype 106 | R | L |
| R23 621 | b | Ribotype 179 | R | L |
| R23 635 | b | Ribotype 015 | R | L |
| R23 639 | b | Ribotype 014 | R | L |
| R23 642 | b | Ribotype 012 | R | L |
| R23 720 | b | Ribotype 005 | R | L |
| R23 727 | b | Ribotype 001 | R | L |
| R23 732 | b | Ribotype 027 | R | L |
| R23 737 | b | Ribotype 106 | R | L |
| G83/03 | b | Ribotype 180 | R | L |
| 12056 | c | Rumen of new-born lamb | nt | L |
| 12057 | c | Rumen of new-born lamb | nt | L |

Sources
a: National Collection of Type Cultures, Central Public Health Laboratory, 61, Colindale Ave, London;
b: Dr Jonathan Brazier, Anaerobe Reference Unit, Dept. of Medical Microbiology and PHLS, University Hospital of Wales, Heath Park, Cardiff;
c: Deutsche Sammlung von Mikroorganismen and Zellkulturen (DSMZ), GmbH Inhoffenstrasse 7 B, 38124 Braunschweig, Germany.
S = shows sensitivity to infection by bacteriophage φDCD27,
R = insensitive to infection by bacteriophage φCD27,
nt = not tested;
L = lysed by φDCD27 lysin.

TABLE 2
Spectrum of activity of φCD27 lysin against a range of bacteria.

| Bacterium | Strain | Lysin test |
|---|---|---|
| Bacillus cereus | ATCC 9139 | ++ |
| Bacillus subtilis | ATCC 6633 | + |
| Bifidobacterium adolescentis | DSMZ 20083 | − |
| Bifidobacterium angulatum | DSMZ 20098 | − |
| Bifidobacterium bifidum | DSMZ 20082 | − |
| Bifidobacterium longum | DSMZ 20219 | − |
| Bifidobacterium pseudocatenulatum | DSMZ 20438 | − |
| Clostridium coccoides | NCTC 11035 | − |
| Clostridium perfringens | NCTC 3110 | − |
| Clostridium bifermentans | C22/10 | +++ |
| Clostridium bifermentans | NCTC 13019 | ++ |
| Clostridium sordelli | NCTC 13356 | ++ |
| Clostridium sporogenes | ATCC 17886 | − |
| Clostridium tyrobutyricum | NCIMB 9582 | − |
| Enterococcus faecalis | FI10734 | − |
| Enterococcus faecium | FI10735 | − |
| Enterococcus hirae | Fl10477 | − |
| Escherichia coli wild type | K12 | − |
| Lactobacillus bulgaricus | FI10643 | − |
| Lactobacillus casei | FI107346 | − |
| Lactobacillus gasseri | NCIMB1171 | − |
| Lactobacillus johnsonii | FI109785 | − |
| Lactobacillus plantarum | FI108595 | − |
| Lactobacillus rhamnosus | Fl107347 | − |
| Lactobacillus sakei | FI10645 | − |
| Lactococcus lactis | MG1316 | − |
| Lactococcus garvieae | FI08174 | − |
| Listeria innocua | NCTC 11288 | − |
| Listeria ivanovii | NCTC 11007 | + |
| Listeria monocytogenes | NCTC 5412 | − |
| Micrococcus luteus | FI106340 | − |
| Pediococcus pentosaceus | FI10642 | − |
| Pediococcus acidilactici | FI10738 | − |
| Salmonella enterica serovar Typhimurium | FI10739 | − |
| Salmonella enteriditis | FI10113 | − |
| Staphylococcus aureus | FI10139 | − |
| Streptococcus anginosus | FI10740 | − |
| Veilonella atypical | FI10741 | − |

− = no lysis,
+++ = clear lysis,
+ = limited lysis

In addition to the above, it is noted that many additional strains representative of commensal strains which are desirably not harmed in order to maintain health gut physiology, are not harmed by contact with the lysin according to this invention. All of the following Clostridium species tested against φCD27, all from DSMZ, gave no lysis. These strains were specifically chosen on the basis of being representative of the main Clostridium clusters commonly found in the human gut, as references Eckberg et al. (2005) Science 308 1635- and supplementary material, and Kikuchi et al., (2002) Microbiol. Immunol. 46, 353 and refs therein:

TABLE 3
GI tract Clostridium and clostridium-like species not lysed by φCD27 lysin.

| Bacterial cells | Deposit | Cluster |
|---|---|---|
| Clostridium cellobioparum | DSMZ 1351 | Cluster III |
| Clostridium leptum | DSMZ 753 | Cluster IV |
| Clostridium nexile | DSMZ 1787 | Cluster XIVa |
| Clostridium colinum | DSMZ 6011 | Cluster XIVb |
| Clostridium innocuum | DSMZ 1286 | Cluster XIVb |
| Clostridium ramosum | DSMZ 1402 | Cluster XVIII |
| Eubacterium barkeri (formally C. barkeri) | DSMZ 1223 | Cluster XV |
| Anaerococcus hydrogenalis | DSMZ 7454 | Cluster XIII |

All C. difficile strains were tested against C. difficile strain 630 prophage 1 lysin expressed in E. coli by the same method and the CD630L1 lysin gave no lysis.

### Cell Viability

To measure the effect of phiCD27 lysin on cell viability, replicate assays were set up under anaerobic conditions using pre-reduced buffers and media. Cells were grown to end log, harvested by centrifugation in anaerobic conditions then resuspended in PBS buffer at pH 7.3. 10-fold dilutions were made in PBS from c. 1×10⁸ cells to c. 1×10³ cells; 10 ul aliquots of these dilutions were themselves serially diluted in PBS from 10⁻¹ to 10⁻⁶ and 10 ul portions of the dilutions were spotted onto BHI agar at time 0 to allow estimation of the number of cells in each assay. Assays were in duplicate and contained either 100 μg partially-purified endolysin (E1) or an equivalent volume (50 μl) of buffer (EB) and cells to a final volume of 300 μl. After 2 h incubation with continuous gentle shaking, 30 μl samples were taken for 10-fold serial dilutions in PBS; 10 μl aliquots of these dilutions were spotted onto BHI agar and the remaining 270 μl assay from one of each duplicate pair was plated to allow cell enumeration.

Assays containing c. 1×10⁸ cells at time 0 showed a drop of 1 log after 2 h incubation, while assays to which 1×10⁷ cells or 1×10⁶ cells had been added showed a drop of 2 log compared to buffer controls. In assays with lower initial cell numbers the lysin was more effective, with only 4 viable colonies being recovered from an assay inoculated with 1×10⁵ cells and no live cells remaining in assays of 1×10⁴ cells or less.

The above viability assay was then repeated using a 400 μl aliquot of E1 that had been subjected to a buffer exchange using 2 ml Zeba Desalt spin columns (Pierce) to replace the Ni-NTA elution buffer (EB) with 20 mM sodium phosphate pH 6 (NP). The lysin in NP buffer showed equivalent activity against frozen cells of Clostridium difficile 11204 to the original NiNTA E1 (FIG. 15). The viability assay was repeated as above using 50 μg E1-NP or NP buffer control and c. 1×10⁶ cells; a 2 h incubation with the lysin produced a drop of 3 log compared to buffer controls.

The above data subsequently formed the basis of a published scientific manuscript (Mayer et al., 2008, J. Bacteria 190:6734-6740; ref no. 39), the disclosures of which are incorporated herein by reference.

### Domain Swapping

Engineering New Enzymatic Domains onto the φCD27L Endolysin by Splice Overlap PCR

The endolysin LM-4 from bacteriophage φLM4, active against Listeria monocytogenes, was demonstrated to cause effective lysis of host cells (see GB 2,255,561 B). The endolysin is 864 by long, giving a protein of 287 amino acids which shows homology to pfam02557, VanY, D-alanyl-D-alanine carboxypeptidase in the first part of the protein and COG5632, N-acetylmuramoyl-L-alanine amidase over the whole sequence (NCBI Blast). The first half of the sequence, encoding the proposed enzyme active domain (EAD), was inserted upstream of either the proposed CD27L cell wall binding domain (CBD, from Asn 180 to the final Arg 270) or the entire 270 amino acid enzyme by splice overlap extension PCR. The LM4 enzymatic domain was amplified by PCR from plasmid pF1567 (Payne et al., 1996 FEMS Microbiology Letters 136: 19-24) using primers LM4Nde 5′-GGA TGA TTA CAT ATG GCA TTA ACA G [SEQ ID NO: 15], to create an NdeI site at the ATG of LM4, and one of two splice overlap primers: LM4-splice-CD27LE 5′-TAT ACA TAT TTT CAT GTT TTG TGT CGC AGT [SEQ ID NO: 16], which represents nucleotides 439-453, Thr147 to Asn 151, of the LM4 sequence with a tail that matches the first 15 nucleotides of the CD27L enzyme to give LM4 EAD-CD27L EAD-CBD; or LM4-splice-CD27L 5′-TTT AAC TCC CTC ATT GTT TTG TGT CGC AGT [SEQ ID NO: 17], which represents nucleotides 439-453, Thr147 to Asn 151, of the LM4 sequence with a tail that matches the proposed C-terminal binding domain of CD27L from Asn 180 to Arg 270, to give LM4 EAD-CD27L CBD. Similarly, the CD27L entire sequence or proposed CBD were amplified from φCD27L-pET15b using a primer from the vector, T7T 5′-GCT AGT TAT TGC TCA GCG G [SEQ ID NO: 18] and splicing primers which had tails to match the end of the LM4 EAD sequence—CD27LEspliceLM4 5′-ACT GCG ACA CAA AAC ATG AAA ATA TGT ATA ACA GT [SEQ ID NO: 19] for the entire sequence, where the last 20 nt of the primer encode the beginning of the CD27L sequence from Met 1; and CD27LspliceLM4 5′-CT GCG ACA CAA AAC AAT GAG GGA GTT AAA C [SEQ ID NO: 20] for the CBD only, where the last 16 nt of the primer encodes the proposed CBD of the CD27L sequence from Asn180. PCR was performed with Phusion (Finnzymes) with the conditions recommended by the manufacturer, using annealing temperatures for 5 cycles to match the portion of the splicing primer which gave 100% match to the original template, then 20 cycles at an annealing temperature to match the entire splicing primer. Products were purified using SureClean (Bioline) and resuspended in a volume of 50 μl. These templates were diluted 100-fold and 1 μl aliquots used in a PCR reaction using the original outer primers—LM4Nde and T7T—at an annealing temperature to allow splicing of the two sequences (54° C.). The final products were purified with SureClean, restricted with NdeI and XhoI and subcloned into pET15b to produce His-tagged LM4-CD27LE and LM4-CD27L. These plasmids were then transformed into E. coli and their sequences confirmed.

Both crude extracts and NiNTA-purified extracts of the composite enzymes were produced, analysed by SDS-PAGE and Western blotting and assayed as described previously (see FIG. 16). Both His-tagged LM4-CD27LE and LM4-CD27L were present at high levels in crude extracts. When incubated with frozen cells of C. difficile 11204 in PBS buffer pH 5.8, 10 μg NiNTA-purified extracts produced a rapid lysis compared to buffer controls (see FIG. 17), with LM4-CD27LE showing a similar speed of lysis to the native CD27L. An equivalent activity was seen using PBS buffer at pH 7.3 as the cell diluent.

In a viability assay, NiNTA-purified eluates of both LM4-CD27LE and LM4-CD27L produced a drop in viable counts (see FIG. 17). Using 50 μg NiNTA E1, assays containing c.1×10⁴ cells showed a reduction of at least 1 log after 2 h incubation compared to buffer controls. This drop was not as great as that seen with the native enzyme, but proves the principle that the addition of alternate enzyme domains can produce active novel enzymes which have the capability to kill C. difficile.

### Nucleotide and Amino Acid Sequences of Wildtype LM4 and Domain Swapped Lysins

(a) LM4
[SEQ ID NO: 21]
ATGGCATTAACAGAGGCATGGCTAATTGAAAAAGCAAATCGCAAATTGAATACGTCA
GGTATGAATAAAGCTACATCTGATAAGACTCGGAATGTAATTAAAAAAATGGCAAAA
GAAGGGATTTATCTTTGTGTTGCGCAAGGTTACCGCTCAACAGCGGAACAAAATGC
GCTATATGCACAAGGGAGAACCAAACCTGGAGCGATTGTTACTAATGCTAAAGGTG
GGCAATCTAATCATAATTTCGGTGTAGCAGTTGATTTGTGCTTGTATACGAGCGACG
GAAAAGATGTTATTTGGGAGTCGACAACTTCCCGGTGGAAAAAGGTTGTTGCTGCT
ATGAAAGCGGAAGGATTCGAATGGGGCGGAGATTGGAAAAGTTTTAAAGACTATCC
GCATTTTGAACTATGTGACGCTGTAAGTGGTGAGAAAATCCCTACTGCGACACAAAA
CACCAATCCAAACAGACATGATGGGAAAATCGTTGACAGCGCGCCACTATTGCCAA
AAATGGACTTTAAATCAAATCCAGCGCGCATGTATAAATCAGGAACTGAGTTCTTAG
TATATGAACATAATCAATATTGGTACAAGACGTACATCAACGACAAATTATACTACAT
GTATAAGAGCTTTTGCGATGTTGTAGCTAAAAAAGATGCAAAAGGACGCATCAAAGT
TCGAATTAAAAGCGCGAAAGACTTACGAATTCCAGTTTGGAATAACACAAAATTGAA
TTCTGGGAAAATTAAATGGTATGCACCCAATACAAAATTAGCATGGTACAACAACGG
AAAAGGATACTTGGAACTCTGGTATGAAAAGGATGGCTGGTACTACACAGCGAACT
ACTTCTTAAAATAA
[SEQ ID NO: 22]
MALTEAWLIEKANRKLNTSGMNKATSDKTRNVIKKMAKEGlYLCVAQGYRSTAEQNALY
AQGRTKPGAIVTNAKGGQSNHNFGVAVDLCLYTSDGKDVIWESTTSRWKKVVAAMKA
EGFEWGGDWKSFKDYPHFELCDAVSGEKIPTATQNTNPNRHDGKIVDSAPLLPKMDFK
SNPARMYKSGTEFLVYEHNQYWYKTYINDKLYYMYKSFCDVVAKKDAKGRIKVRIKSAK
DLRIPVWNNTKLNSGKIKWYAPNTKLAWYNNGKGYLELWYEKDGWYYTANYFLK
(b) LM4-CD27LE
[SEQ ID NO: 23]
ATGGCATTAACAGAGGCATGGCTAATTGAAAAAGCAAATCGCAAATTGAATACGTCA
GGTATGAATAAAGCTACATCTGATAAGACTCGGAATGTAATTAAAAAAATGGCAAAA
GAAGGGATTTATCTTTGTGTTGCGCAAGGTTACCGCTCAACAGCGGAACAAAATGC
GCTATATGCACAAGGGAGAACCAAACCTGGAGCGATTGTTACTAATGCTAAAGGTG
GGCAATCTAATCATAATTTCGGTGTAGCAGTTGATTTGTGCTTGTATACGAGCGACG
GAAAAGATGTTATTTGGGAGTCGACAACTTCCCGGTGGAAAAAGGTTGTTGCTGCT
ATGAAAGCGGAAGGATTCGAATGGGGCGGAGATTGGAAAAGTTTTAAAGACTATCC
GCATTTTGAACTATGTGACGCTGTAAGTGGTGAGAAAATCCCTACTGCGACACAAAA
CATGAAAATATGTATAACAGTAGGACACAGTATTTTAAAAAGTGGAGCATGTACTTCT
GCTGATGGAGTAGTTAACGAGTATCAATACAACAAATCTCTTGCACCAGTATTAGCA
GATACATTTAGAAAAGAAGGGCATAAGGTAGATGTAATAATATGCCCAGAAAAGCAG
TTTAAAACTAAGAATGAAGAAAAGTCTTATAAAATACCTAGAGTTAATAGTGGAGGAT
ATGATTTACTTATAGAGTTACATTTAAATGCAAGTAACGGTCAAGGTAAAGGTTCAGA
AGTCCTATATTATAGTAATAAAGGCTTAGAGTATGCAACTAGAATATGTGATAAACTA
GGTACAGTATTTAAAAATAGAGGTGCTAAATTAGATAAAAGATTATATATCTTAAATA
GTTCAAAGCCTACAGCAGTATTAATTGAAAGTTTCTTCTGTGATAATAAAGAAGATTA
TGATAAAGCTAAGAAACTAGGTCATGAAGGTATTGCTAAGTTAATTGTAGAAGGTGT
ATTAAATAAAAATATAAATAATGAGGGAGTTAAACAGATGTACAAACATACAATTGTT
TATGATGGAGAAGTTGACAAAATCTCTGCAACTGTAGTTGGTTGGGGTTATAATGAT
GGGAAAATACTGATATGTGATATAAAAGATTACGTGCCAGGTCAGACGCAAAATCTT
TATGTTGTAGGAGGTGGCGCATGTGAAAAGATAAGTTCTATTACTAAAGAAAAATTT
ATTATGATAAAAGGTAATGATAGATTTGATACACTTTATAAAGCATTGGATTTTATTAA
TAGATAG
[SEQ ID NO: 24]
MALTEAWLIEKANRKLNTSGMNKATSDKTRNVIKKMAKEGIYLCVAQGYRSTAEQNALY
AQGRTKPGAIVTNAKGGQSNHNFGVAVDLCLYTSDGKDVIWESTTSRWKKVVAAMKA
EGFEWGGDWKSFKDYPHFELCDAVSGEKIPTATQNMKICITVGHSILKSGACTSADGVV
NEYQYNKSLAPVLADTFRKEGHKVDVIICPEKQFKTKNEEKSYKIPRVNSGGYDLLIELH
LNASNGQGKGSEVLYYSNKGLEYATRICDKLGTVFKNRGAKLDKRLYILNSSKPTAVLIE
SFFCDNKEDYDKAKKLGHEGIAKLIVEGVLNKNINNEGVKQMYKHTIVYDGEVDKISATV
VGWGYNDGKILICDIKDYVPGQTQNLYVVGGGACEKISSITKEKFIMIKGNDRFDTLYKAL
DFINR
(c) LM4-CD27L
[SEQ ID NO: 25]
ATGGCATTAACAGAGGCATGGCTAATTGAAAAAGCAAATCGCAAATTGAATACGTCA
GGTATGAATAAAGCTACATCTGATAAGACTCGGAATGTAATTAAAAAAATGGCAAAA
GAAGGGATTTATCTTTGTGTTGCGCAAGGTTACCGCTCAACAGCGGAACAAAATGC
GCTATATGCACAAGGGAGAACCAAACCTGGAGCGATTGTTACTAATGCTAAAGGTG
GGCAATCTAATCATAATTTCGGTGTAGCAGTTGATTTGTGCTTGTATACGAGCGACG
GAAAAGATGTTATTTGGGAGTCGACAACTTCCCGGTGGAAAAAGGTTGTTGCTGCT
ATGAAAGCGGAAGGATTCGAATGGGGCGGAGATTGGAAAAGTTTTAAAGACTATCC
GCATTTTGAACTATGTGACGCTGTAAGTGGTGAGAAAATCCCTACTGCGACACAAAA
CAATGAGGGAGTTAAACAGATGTACAAACATACAATTGTTTATGATGGAGAAGTTGA
CAAAATCTCTGCAACTGTAGTTGGTTGGGGTTATAATGATGGGAAAATACTGATATG
TGATATAAAAGATTACGTGCCAGGTCAGACGCAAAATCTTTATGTTGTAGGAGGTGG
CGCATGTGAAAAGATAAGTTCTATTACTAAAGAAAAATTTATTATGATAAAAGGTAAT
GATAGATTTGATACACTTTATAAAGCATTGGATTTTATTAATAGATAG
[SEQ ID NO: 26]
MALTEAWLIEKANRKLNTSGMNKATSDKTRNVIKKMAKEGIYLCVAQGYRSTAEQNALY
AQGRTKPGAIVTNAKGGQSNHNFGVAVDLCLYTSDGKDVIWESTTSRWKKVAAMKAE
GFEWGGDWKSFKDYPHFELCDAVSGEKIPTATQNNEGVKQMYKHTIVYDGEVDKISAT
VVGWGYNDGKILICDIKDYVPGQTQNLYVVGGGACEKISSITKEKFIMKGNDRFDTLYKA
LDFINR

### Example B

### Exemplary Truncation Variants of the Endolysin of SEQ ID NO:1

### Methods

CD27L truncations CD27L₁₋₁₇₉ (“27*”) and CD27L₁₈₀₋₂₇₀(“27”) were produced by PCR from construct CD27L-pET15b (39) using GoTaq Polymerase (Promega).

CD27L₁₋₁₇₉(“27*”)
[SEQ ID NO: 2]
MKICITVGHSILKSGACTSADGVVNEYQYNKSLAPVLADTFRKEGHKVDVIICPEKQFKTKNEEKS
YKIPRVNSGGYDLLIELHLNASNGQGKGSEVLYYSNKGLEYATRICDKLGTVFKNRGAKLDKRLYI
LNSSKPTAVLIESFFCDNKEDYDKAKKLGHEGIAKLIVEGVLNKNIN
CD27L₁₈₀₋₂₇₀(“*27”)
[SEQ ID NO: 27]
MNEGVKQMYKHTIVYDGEVDKISATVVGWGYNDGKILICDIKDYVPGQTQNLYVVGGGACEKISS
ITKEKFIMIKGNDRFDTLYKALDFINR

Truncation “27*” (CD27L₁₋₁₇₉; corresponding to amino acids 1 to 179 of SEQ ID NO:1; predicted enzyme active domain, EAD) was amplified using primer T7P (5′-TAA TAC GAC TCA CTA TAG GG) [SEQ ID NO: 28] from the pET15b vector and a primer to create a stop codon after Asn 179 of SEQ ID NO:1, CD27L EAD (5′-TTA ACT CCC TCC TAA TTT ATA TT [SEQ ID NO: 29], altered nucleotides given in bold) giving a 698 by product.

Truncation “*27” (CD27L₁₈₀₋₂₇₀; corresponding to amino acids 180 to 270 of SEQ ID NO:1) was amplified to start from Asn 180 of SEQ ID NO:1, using a primer to create an NdeI site encompassing an initiation Met immediately upstream of Asn 180-CD27L_CBD (5′-CAT ATG AAT GAG GGA GTT AAA CAG ATG) [SEQ ID NO: 30] paired with T7T from the pET15b vector (5′-GCT AGT TAT TGC TCA GCG G) [SEQ ID NO: 18], giving 370 bp.

PCR conditions were as described (Promega) using annealing temperatures of 42° C. for 5 cycles followed by 20 cycles at 46° C. for 27* and 46° C. for 25 cycles for *27, with an extension time of 40 s. Both products were subcloned into pCR2.1 using the TA cloning kit (Invitrogen) and FastLink DNA ligase (Epicentre). The constructs were transformed into chemically competent E. coli TOP10 cells (Invitrogen) for sequence and orientation confirmation. Products were excised with NdeI and XhoI, subcloned into pET15b which had been restricted with NdeI and XhoI and dephosphorylated with Antarctic Phosphatase (Promega), and transformed for sequence confirmation as before. For expression, constructs were transformed into chemically competent E. coli BL21(DE3) cells (Invitrogen).

The exemplary truncation mutants, “27*” and “*27”, were thus produced with a His tag amino acid sequence (MGSSHHHHHHSSGLVPRGSH; [SEQ ID NO: 3]) at their amino terminus; see SEQ ID NO: 4 above.

Crude and NiNTA-purified protein extracts were produced and assayed for lytic activity against fresh and frozen C. difficile cells as described (39) Partially-purified extracts were also produced using Ni-NTA spin columns in native buffer as described by the manufacturer (Qiagen) so that samples could be extracted and assayed contemporaneously. Cells of C. difficile NCTC 11204 were grown from fresh cultures in BHI+C to end log and harvested by centrifugation (39). Lysis was monitored by drop in optical density as described previously, with mutanolysin (Sigma) as a positive control. Species selected from Tables 1-3 were grown similarly in BHI+C (Clostridiales), RCM (Oxoid, C. tyrobutyricum) or GM17 (L. lactis) to end log or stationary phase and harvested by centrifugation. Cell viability assays were conducted as described previously (39) using NiNTA-purified protein in elution buffer. Assessment of endolysin activity in media was conducted in 3 ml Bijoux of BHI+C under anaerobic conditions with enumeration of C. difficile cells on CCEY agar.

### Results

In a variety of tests on fresh and frozen cells of C. difficile strain NCTC 11204, the “27*” truncated endolysin from crude protein extracts and Ni-NTA purified samples consistently effected more rapid lysis than the native endolysin (FIG. 18a). The “27*” truncated endolysin [SEQ ID NO:4] was active against all 32 strains listed in Table 1; in all cases “27*” caused more rapid lysis than the native CD27L enzyme when the proteins were added at equivalent concentrations (data not shown).

The lysis observed from extracts containing the C-terminal half of CD27L, “*27”, was similar to that seen with buffer or empty vector controls.

Like the native endolysin, “27*” showed no lytic activity on a range of other Clostridial species including C. tyrobutyricum (FIG. 18b), Anaerococcus hydrogenalis, C. cellobioparum, C. coccoides, C. innocuum, C. perfringens, C. ramosum, C. sporogenes, Eubacterium barkeri or on Lactococcus lactis, indicating that a measure of specificity is retained in the truncated form (data not shown).

The effect of the native and truncated endolysins on cell viability was tested using 100 μM CD27L or “27*” (equivalent to 100 μg CD27L). In assays containing c. 5×10⁴ cfu, CD27L reduced surviving cell numbers to 109 and 66 cfu in each of the duplicate assays; as noted previously, in assays containing c. 5×10⁵ cfu, cell numbers were reduced by 2 log. In the same assays truncated endolysin “27*” was much more effective, with no live cells being recovered. At higher cell densities of c. 1×10⁸ or 1×10⁷ cfu CD27L gave reductions of c. 0.5 log and c. 1 log respectively, while “27*” was more effective, giving a reduction of more than 4-log from 1×10⁸ cfu (to c. 5.8×10³ cfu) and an almost 6-log drop of 1×10⁷ cfu to c. 20 cfu with both 100 μM and 50 μM endolysin; those colonies that survived the endolysin treatment showed a slow growth phenotype requiring 2 d incubation.

Cell viability assays investigating the effect of CD27L in BHI+C media consistently failed to demonstrate cell killing, possibly due to compounds in the media affecting enzyme activity or cell lysis. The truncated “27*” endolysin did however show a bacteriostatic effect in medium. When 1000 μg NiNTA-purified “27*” in elution buffer was incubated with c. 1×10⁵ cfu in 3 ml BHI+C this cell density was maintained throughout the sampling times at 2, 4, 6, 8 and 24 h; in contrast, cells in the elution buffer control multiplied to almost 1×10⁸ cfu at 8 h, although the viable cell numbers had returned to 1×10⁵ cfu by 24 h (FIG. 19). No live cells were recovered from an assay conducted in parallel in PBS buffer containing 1000 μg of the same lysin preparation and 1×10⁴ cfu incubated for 2 h. After 24 h, 30 μl of the 3 ml assay (representing c. 10 μg endolysin) was tested in a standard lysis assay with fresh C. difficile cells. This demonstrated that the endolysin that had been incubated in media for 24 h at 37° C. showed equivalent activity to a sample that had been stored in elution buffer at 4° C.

### Example C

### Exemplary Substitution Variants of the Endolysin of SEQ ID No:1

### Methods

Cysteine mutants were created from the endolysin of SEQ ID NO:1 subcloned into the NdeI-XhoI sites of pET15b (39) using splice overlap PCR and Phusion polymerase (Finnzymes).

To create “CM1”/“CYSMUT1” (Cys 53 TGC to Ser 53 AGC), two PCR products were produced using primer T7P (5′-TAA TAC GAC TCA CTA TAG GG) [SEQ ID NO: 28] from the pET15b vector and CD27L_{—}4 (5′-CTG GGC TTA TTA TTA CAT CT [SEQ ID NO: 31], altered nucleotides given in bold, see FIG. 20), and from CD27L_{—}3 (5′-GTA ATA ATA AGC CCA GAA AAG C) [SEQ ID NO: 32] and CD27L_{—}6 (5′-TGT CAA CTT CTC CAT CAT) [SEQ ID NO: 33]. These two products were spliced and amplified with T7P and CD27L_{—}6 to give a 742 by product. The product was restricted with NdeI and MunI and used to replace the NdeI-MunI fragment of CD27L-pET15b.

To create “CM2”/“CYSMUT2” (Cys 217 to Ser 217), PCR products were produced using primer pair CD27L-5 (5′-GAG GGA GTT AAA CAG ATG TA) [SEQ ID NO: 34] and CD27L_{—}8 (5′-TAT ATC ACT TAT CAG TAT TTT CC) [SEQ ID NO: 35] and primer pair CD27L-7 (5′-CTG ATA ATG GAT ATA AAA GAT TAC) [SEQ ID NO: 36] and T7T from the pET15b vector (5′-GCT AGT TAT TGC TCA GCG G) [SEQ ID NO: 18]. These products were spliced and amplified with CD27L-5 and T7T and the resulting 361 bp product was restricted with MunI and XhoI and used to replace the MunI-XhoI fragment of CD27L-pET15b.

Finally, “CM3”/“CYSMUT3” (Cys 238 to Ser 238) was engineered in the same way using primer pair CD27L_{—}5 and CD27L_{—}10 (5′-CCA CCG CGT ACA CTT TTC) [SEQ ID NO: 37] and CD27L_{—}9 (5′-GGC GCA AGT GM AAG ATA AG) [SEQ ID NO: 38] paired with T7T.

All three constructs were transformed into E. coli TOP10 chemically competent cells (Invitrogen) for sequence confirmation then into E. coli BL21(DE3) chemically competent cells (Invitrogen) for protein expression, as described previously (39).

Crude and NiNTA-purified protein extracts were produced and assayed for lytic activity against fresh and frozen C. difficile cells as described previously (39). Partially-purified extracts were also produced using Qiagen Ni-NTA spin columns so that samples could be extracted and assayed contemporaneously.

### Results

In a variety of tests on fresh and frozen cells the CM1 to 3 mutant endolysins from crude protein extracts and Ni-NTA purified samples showed similar activity to the native endolysin (FIG. 21).

However, oligomerisation was greatly reduced in CM1, which allowed crystal formation (data not shown).

### Example D

### Expression and Delivery of Truncation Variants of the Endolysin of SEQ ID NO:1 in Lactococcus lactis

### Methods

Three sequences were investigated for expression and delivery in L. lactis: endolysin CD27L [SEQ ID NO: 1], truncation variant “27*” [see SEQ ID NO: 2 above, CD27L₁₋₁₇₉] and “CM1” [SEQ ID NO: 39, CD27L Cys53Ser].

CM1
[SEQ ID NO: 39]
MKICITVGHSILKSGACTSADGVVNEYQYNKSLAPVLADTFRKEGHKVDVIISPEKQFKT
KNEEKSYKIPRVNSGGYDLLIELHLNASNGQGKGSEVLYYSNKGLEYATRICDKLGTVFK
NRGAKLDKRLYILNSSKPTAVLIESFFCDNKEDYDKAKKLGHEGIAKLIVEGVLNKNINNE
GVKQMYKHTIVYDGEVDKISATVVGWGYNDGKILICDIKDYVPGQTQNLYVVGGGACEK
ISSITKEKFIMIKGNDRFDTLYKALDFINR

“27” and “cm1” templates were amplified by PCR from constructs 27*-pET15b and CM1-pET15b using primers CD27LCOD2_F [SEQ ID NO: 13] and T7T [SEQ ID NO: 18] to amplify the endolysin sequences from the second codon and some downstream vector sequence which included a BamHI restriction site after the lysin coding sequence (see FIG. 5). Amplification was as described previously using Phusion DNA polymerase with an annealing temperature of 56° C. The purified PCR products were restricted with BamHI and ligated into the vector pUK200His which had been restricted with NcoI and end filled with T4 DNA polymerase (Promega) then restricted with BamHI. This put the endolysin sequences after an in-frame His tag MSHHHHHHA [SEQ ID NO: 5]. Cd27l-pUK200His was already available (Mayer et al., 2008). These three constructs were used to produce His-endolysin templates to be spliced to templates encoding PnisA-SLPmod—the nisinA promoter and novel signal peptide described in Fernandez et al. (40)—produced from construct pFI2596 of the same reference, wherein PnisA-SLPmod-IL12 is subcloned into the EcoRI site of plasmid pTG262. Splice template PnisA-SLPmod was produced from pFI2596 using primers pTG262_{—}2 (5′-CAG GTC GAC TCT AGA GGA TCC) [SEQ ID NO: 40] and SLPSPLICEHIS (5′-ATG GTG ATG ACT CAT AGC AGC ATT AAC TGG) [SEQ ID NO: 41] using annealing temperatures of 41° C. for 5 cycles and 62° C. for 20 cycles and an extension time of 10s to give the 409 by product. Splice templates his-cd271, his-cm1 and his-27* were produced from cd27l-pUK200His, cm1-pUK200His and 27*-pUK200His respectively using primers HISSPLICESLP (5′-CCA GTT AAT GCT ATG AGT CAT CAC CAT CA) [SEQ ID NO: 42] and p181 (5′-GCG AAG ATA ACA GTG ACT CTA) [SEQ ID NO: 43] which amplified the His-tagged endolysins and some downstream pUK200His vector sequence including an EcoRI site. Amplification used annealing temperatures of 42° C. for 5 cycles and 59° C. for 20 cycles and an extension time of 20s to give the 907 by product (his-cd271, his-cm1) or 15s for the 672 by his-27* product. These three products were combined in separate reactions with splice template PnisA-SLPmod (see SEQ ID NO:44 below) using DNA concentrations of 1-4 ng per template and spliced products were amplified using primers pTG262_{—}2 [SEQ ID NO: 40] and p181 [SEQ ID NO: 43] with an annealing temperatures of 59° C. and an extension time of 30 s to give the 1278 by (his-cd271, his-cm1) and 1040 by (his-27*) products. Purified products were restricted with EcoRI and ligated into EcoRI-restricted, Antarctic phosphatase-treated pTG262. Ligation products were transformed into electrocompetent E. coli MC1022 and positive transformants were selected on chloramphenicol (15 μg/ml). Constructs PnisASLPmodHiscd271-pTG262, PnisASLPmodHiscm1-pTG262 and PnisASLPmodHis27*pTG262 (see SEQ ID NOS:45 to 47 below, respectively) were confirmed by sequence analysis then transformed, along with the vector control pTG262, into electrocompetent L. lactis FI5876 (40) which is a constitutive nisin producer. Positives transformants were selected on GM17 containing 5 μg/ml chloramphenicol.

### PnisA-SLPmod Nucleotide Sequence (Positioned Directly Upstream of ATG of Endolysin Sequences)

[SEQ ID NO: 44]
AAACGGCTCTGATTAAATTCTGAAGTTTGTTAGATACAATGATTTCGTTC
GAAGGAACTACAAAATAAATTATAAGGAGGCACTCAAAATGGGTAAAAAA
AATTTAAGAATTGTTAGTGCTGCTGCTGCTGCTTTATTAGCTGTTGCTCC
AGTTGCTGCAACTGCTATGCCAGTTAATGCTGCTATGAGTCATCACCATC
ACCATCACGCC
Key: PnisA = underlined, RBS = double-
underlined, SLPmod = italics, His tag = bold

### PnisASLPmodHiscd271 Amino Acid Sequence

[SEQ ID NO: 45]
MGKKNLRIVSAAAAALLAVAPVAATAMPVNAAMSHHHHHHAMKICITVGHSILKSGACT
SADGVVNEYQYNKSLAPVLADTFRKEGHKVDVIICPEKQFKIKNEEKSYKIPRVNSGGY
DLLIELHLNASNGQGKGSEVLYYSNKGLEYATRICDKLGTVFKNRGAKLDKRLYILNSSK
PTAVLIESFFCDNKEDYDKAKKLGHEGIAKLIVEGVLNKNINNEGVKQMYKHTIVYDGEV
DKISATVVGWGYNDGKILICDIKDYVPGQTQNLYVVGGGACEKISSITKEKFIMIKGNDRF
DTLYKALDFINR

### PnisASLPmodHiscm1 Amino Acid Sequence

[SEQ ID NO: 46]
MGKKNLRIVSAAAAALLAVAPVAATAMPVNAAMSHHHHHHAMKICITVGHSILKSGACT
SADGVVNEYQYNKSLAPVLADTFRKEGHKVDVIISPEKQFKTKNEEKSYKIPRVNSGGY
DLLIELHLNASNGQGKGSEVLYYSNKGLEYATRICDKLGTVFKNRGAKLDKRLYILNSSK
PTAVLIESFFCDNKEDYDKAKKLGHEGIAKLIVEGVLNKNINNEGVKQMYKHTIVYDGEV
DKISATVVGWGYNDGKILICDIKDYVPGQTQNLYVVGGGACEKISSITKEKFIMIKGNDRF
DTLYKALDFINR

### PnisASLPmodHis27* Amino Acid Sequence

[SEQ ID NO: 47]
MGKKNLRIVSAAAAALLAVAPVAATAMPVNAAMSHHHHHHAMGSSHHHHHHSSGLVP
RGSHMKICITVGHSILKSGACTSADGVVNEYQYNKSLAPVLADTFRKEGHKVDVIICPEK
QFKTKNEEKSYK1PRVNSGGYDLLIELHLNASNGQGKGSEVLYYSNKGLEYATRICDKL
GTVFKNRGAKLDKRLYILNSSKPTAVLIESFFCDNKEDYDKAKKLGHEGIAKLIVEGVLNK
NIN

To assess lysin production, L. lactis cultures were grown in 10 ml or 100 ml GM17 broth containing 5 μg/ml chloramphenicol at 30° C. for 6 h. Cells and supernatant were harvested by centrifugation. Proteins were extracted by bead beating into NP buffer as described previously (39). Supernatants were concentrated using Amicon Ultra-4 centrifugal filters (nominal molecular weight limit 10 K for FI5876-PnisASLPmodHis27*-pTG262 or 30 K for FI5876-PnisASLPmodHiscd27l-pTG262 and FI5876-PnisASLPmodHiscm1pTG262; Fisher Scientific). These typically concentrated samples by c. 20×. The lytic activity of supernatants was assessed using plates of 1% agar in 100 ml PBS incorporating autoclaved C. difficile 11204 cells. These were obtained from a 100 ml overnight culture, autoclaved then centrifuged to pellet the cells which were then resuspended in 0.5 ml PBS and added to 100 ml molten agar. Holes were made in the agar using a Pasteur pipette or cork borer and filtered or concentrated supernatant was added. Plate assays were incubated overnight at 37° C. Lytic activity produced from growing cells was also measured using GM17 plates incorporating 5 μg/ml chloramphenicol and autoclaved C. difficile 11204 cells as above. L. lactis from overnight cultures was streaked to single colonies on these plates and incubated 2 d at 30° C. to observe lysis.

### Results

L. lactis strains expressing the constructs PnisASLPmodHiscd271-pTG262, PnisASLPmodHiscm1-pTG262, PnisASLPmodHis27*pTG262 and the empty vector control pTG262 were assessed for externalisation of lytic activity. Both crude protein preparations and concentrated supernatants of all three strains caused lysis of fresh C. difficile cells in turbidity reduction assays; however, the pTG262 control strain also produced a certain amount of lysis due to the production of nisin by strain FI5876. Consequently, lysis was measured on solid media using autoclaved cells. Colonies of FI5876-PnisASLPmodHis27*-pTG262 produced a clear zone of lysis when grown on GM17 agar supplemented with chloramphenicol and autoclaved C. difficile cells. This zone was visible after 2 d incubation and increased upon further incubation (FIG. 22a). Unconcentrated filtered supernatant from FI5876-PnisASLPmodHis27*-pTG262 produced a zone of inhibition of c. 2 mm from 180 μl after overnight incubation at 37° C. Similarly, a c. 20-fold concentrated supernatant from FI5876-PnisASLPmodHis27*-pTG262 produced a c. 3 mm halo of lysis on PBS agar plates incorporating autoclaved C. difficile cells, visible after overnight incubation (FIG. 22b). A larger halo was seen from crude protein extract (FIG. 22c). Colonies and supernatants of strains expressing constructs PnisASLPmodHiscd271-pTG262 or PnisASLPmodHiscm1-pTG262 or the pTG262 control consistently failed to produce visible lysis in plate assays. This effect could reflect the higher activity of the truncated endolysin, or that the smaller protein diffuses more effectively through the agar.

### Example E

### Assessment of Cell Wall Binding Using GFP-Tagged Derivatives

### Methods

The sequence for a red-shifted variant GFP mutant 3 [SEQ ID NO: 48; see below] was subcloned with CD27L, “27*” and “*27” in pET15b to produce His-tagged N-terminally labelled proteins. To facilitate subcloning, an internal NdeI site in GFP3 was first removed by splice overlap PCR, to alter T231 to C231. For this, GFP3 was amplified by PCR from pSB2030 (41) in two parts using primers GFP_NDE (5′-GGA ATA ACA TAT GAG TAA AGG CGA AG, altered nucleotides given in bold) [SEQ ID NO: 49] to create an NdeI site around the start Met codon, and GFPSPLICEGTG (5′-TTT CAT GTG ATC TGG GTA TCT CGC) [SEQ ID NO: 50 to produce a 247 by product and primers GFPSPLICECAC (5′-CCA GAT CAC ATG AAA CAG CAT GAC) [SEQ ID NO: 51] and GFP_TAC (5′-GTA TTT GTA TAG TTC ATC CAT GGC) [SEQ ID NO: 52] to change the TAA stop site to TAC, giving a 495 by product. PCR was performed with Phusion polymerase using conditions as stated before with an extension time of 10 s and annealing temperatures adjusted for the primers (50° C. for 5 cycles and 60° C. for a further 20 cycles for GFPSPLICECAC/GFP_TAC; 50° C. for 10 cycles and 60° C. for a further 20 cycles for GFP_NDE/GFPSPLICEGTG). These two products were purified and spliced together by amplification with primers GFP_NDE [SEQ ID NO: 49] and NGFP_LINK, which overlaps the end of the coding sequence and adds a linker to code for 7 Gly and Ser residues, to allow efficient protein folding, followed by an NdeI site and a EcoRI site to facilitate future subcloning (5′-GGA TGA ACT ATA CAA ATA CGG TAG TGG ATC AGG TAG TGG ACA TAT GAA TTC T, linker given in bold) [SEQ ID NO: 53]. Annealing temperatures were 5 cycles of 37° C. and 20 cycles of 56° C. and an extension time of 30s to give the expected 760 bp product. The PCR product was given 3′ A-overhangs using GoTaq polymerase (Promega) and subcloned into pCR2.1 (Invitrogen) for sequence confirmation. The modified GFP-linker was restricted from this construct using NdeI and subcloned into vector pET15b and constructs CD27L-pET15b, 27*-pET15b and *27-pET15b, all of which had been restricted with NdeI and dephosphorylated with Antarctic phosphatase (New England Biolabs). Ligations were transformed into E. coli TOP10 cells and constructs were sequenced to confirm the correct orientation of His-GFP-endolysin then transformed into E. coli BL21(DE3) for expression.

His-tagged GFP-endolysin fusions were expressed from IPTG-induced E. coli and partially purified using Qiagen Fast-Start NiNTA columns. Column eluates were visibly fluorescent green. Binding of GFP-labelled endolysins to cells of C. difficile was assessed using an adaptation of the method of Loessner et al. (19). Stationary phase cells of C. difficile 11204 were harvested by centrifugation in 1 ml aliquots (2 min at 13,000×g) then resuspended in 1/10 volume PBS-T (PBS pH 7.4 with 0.01% Tween 20) and kept on ice. 100 μl cells were mixed with 100 μl of a 4 μM or 15 μM stock of NiNTA-purified protein and the samples were incubated for 20 min at 37° C. The cells were then pelleted again by centrifugation and washed twice with 500 μl PBS-T by vortexing and centrifugation. Cells were resuspended in 50 μl PBS-T and viewed by fluorescence microscopy using an Olympus BX60 (Olympus, Japan) microscope with ProgRes® Capture Pro 2.1 software (Jenoptik, Germany) and the 100× oil immersion lens. The fluorescence was recorded using the NB filter cube (U-MNB, exciter filter BP470-490, barrier filter BA515). The same samples were also used to perform turbidity reduction assays.

### Results

GFP-labelled CD27L and “27*” showed clear strong binding to cell walls of C. difficile 11204 (FIG. 23a, b). The GFP-labelled C-terminal domain “*27” was also able to bind to cell walls (FIG. 23c) but the number of cells labelled was consistently lower than that seen with GFP-CD27L and GFP-“27*” at equivalent concentrations. No labelling was seen with GFP-pET15b. The method was also successful using lower concentrations of labelled proteins (0.2 μM, 2 μM) and shorter incubation times (5, 10, 15 mins at 37° C.) but labelling increased with both protein concentration and time. Lysis of cells was visible in samples containing GFP-CD27L and GFP-“27*” and the activity of these protein samples was confirmed by turbidity reduction assays (FIG. 23d). The activity was lower than that seen in the unlabelled endolysins. No lytic activity was observed from GFP-“*27” or GFP-pET15b.

GFP3
[SEQ ID NO: 48]
ATGAGTAAAGGCGAAGAACTTTTCACTGGAGTTGTCCCAATTCTTGTTGA
ATTAGATGGTGATGTTAATGGGCACAAATTTTCTGTCAGTGGAGAGGGTG
AAGGTGATGCAACATACGGAAAACTTACCCTTAAATTTATTTGCACTACT
GGAAAACTACCTGTTCCATGGCCAACACTTGTCACTACTTTCGGTTATGG
TGTTCAATGCTTTGCGAGATACCCAGATCATATGAAACAGCATGACTTTT
TCAAGAGTGCCATGCCCGAAGGTTATGTACAGGAAAGAACTATATTTTTC
AAAGATGACGGGAACTACAAGACACGTGCTGAAGTCAAGTTTGAAGGTGA
TACCCTTGTTAATAGAATCGAGTTAAAAGGTATTGATTTTAAAGAAGATG
GAAACATTCTTGGACACAAATTGGAATACAACTATAACTCACACAATGTA
TACATCATGGCAGACAAACAAAAGAATGGAATCAAAGTTAACTTCAAAAT
TAGACACAACATTGAAGATGGAAGCGTTCAACTAGCAGACCATTATCAAC
AAAATACTCCAATTGGCGATGGCCCTGTCCTTTTACCAGACAACCATTAC
CTGTCCACACAATCTGCCCTTTCGAAAGATCCCAACGAAAAGAGAGACCA
CATGGTCCTTCTTGAGTTTGTAACAGCTGCTGGGATTACACATGGCATGG
ATGAACTATACAAATAA

### REFERENCES

1. Kuijper, E. J., Coignard, B. & Tull, P. (2006) Clin Microbiol Infect 12 Suppl 6, 2-18.
2. Anonymous (2006) Health Statistics Quarterly 30, 56-60.
3. Rupnik, M., Dupuy, B., Fairweather, N. F., Gerding, D. N., Johnson, S., Just, I., Lyerly, D. M., Popoff, M. R., Rood, J. I., Sonenshein, A. L., Thelestam, M., Wren, B. W., Wilkins, T. D. & von Eichel-Streiber, C. (2005) J Med Microbiol 54, 113-7.
4. Braun, V., Hundsberger, T., Leukel, P., Sauerborn, M. & von Eichel-Streiber, C. (1996) Gene 181, 29-38.
5. Goncalves, C., Decre, D., Barbut, F., Burghoffer, B. & Petit, J. C. (2004) J Clin Microbiol 42, 1933-9.
6. Popoff, M. R., Rubin, E. J., Gill, D. M. & Boquet, P. (1988) Infect Immun 56, 2299-306.
7. Skurnik, M. & Strauch, E. (2006) Int J Med Microbiol 296, 5-14.
8. Projan, S. (2004) Nat Biotechnol 22, 167-8.
9. Brussow, H., Canchaya, C. & Hardt, W. D. (2004) Microbiol Mol Biol Rev 68, 560-602.
10. Davis, B. M. & Waldor, M. K. (2003) Curr Opin Microbiol 6, 35-42.
11. Strauch, E., Schaudinn, C. & Beutin, L. (2004) Infect Immun 72, 7030-9.
12. Chibani-Chennoufi, S., Sidoti, J., Bruttin, A., Kutter, E., Sarker, S. & Brussow, H. (2004) Antimicrob Agents Chemother 48, 2558-69.
13. Matsuzaki, S., Yasuda, M., Nishikawa, H., Kuroda, M., Ujihara, T., Shuin, T., Shen, Y., Jin, Z., Fujimoto, S., Nasimuzzaman, M. D., Wakiguchi, H., Sugihara, S., Sugiura, T., Koda, S., Muraoka, A. & Imai, S. (2003) J Infect Dis 187, 613-24.
14. Biswas, B., Adhya, S., Washart, P., Paul, B., Trostel, A. N., Powell, B., Carlton, R. & Merril, C. R. (2002) Infect Immun 70, 204-10.
15. Loc Carrillo, C., Atterbury, R. J., el-Shibiny, A., Connerton, P. L., Dillon, E., Scott, A. & Connerton, I. F. (2005) Appl Environ Microbiol 71, 6554-63.
16. Huff, W. E., Huff, G. R., Rath, N. C., Balog, J. M. & Donoghue, A. M. (2004) Poult Sci 83, 1944-7.
17. Ramesh, V., Fralick, J. A. & Rolfe, R. D. (1999) Anaerobe 5, 69-78.
18. Wray, T. (2007) National Provisioner 5th July
19. Loessner, M. J., Kramer, K., Ebel, F. & Scherer, S. (2002) Mol Microbiol 44, 335-49.
20. Loessner, M. J. (2005) Curr Opin Microbiol 8, 480-7.
21. Gasson (1995-2003) Patents GB 2255561 B (1995); AU 650737B (1994); U.S. Pat. No. 5,763,251 (1998); U.S. Pat. No. 6,083,684 (2000); CA 2066387 (2003); EP 0510907B (2003).
22. Fischetti, V. A. (2005) Trends Microbiol 13, 491-6.
23. Schuch, R., Nelson, D. & Fischetti, V. A. (2002) Nature 418, 884-9.
24. Loeffler, J. M., Djurkovic, S. & Fischetti, V. A. (2003) Infect Immun 71, 6199-204.
25. Yoong, P., Schuch, R., Nelson, D. & Fischetti, V. A. (2004) J Bacteriol 186, 4808-12.
26. Zimmer, M., Vukov, N., Scherer, S. & Loessner, M. J. (2002) Appl Environ Microbiol 68, 5311-7.
27. Sell, T. L., Schaberg, D. R. & Fekety, F. R. (1983) J Clin Microbiol 17, 1148-52.
28. Nelson, D. (2004) J Bacteriol 186, 7029-31.
29. Mahony, D. E., Bell, P. D. & Easterbrook, K. B. (1985) J Clin Microbiol 21, 251-4.
30. Goh, S., Riley, T. V. & Chang, B. J. (2005) Appl Environ Microbiol 71, 1079-83.
31. Govind, R., Fralick, J. A. & Rolfe, R. D. (2006) J Bacteriol 188, 2568-77.
32. Goh, S., Ong, P. F., Song, K. P., Riley, T. V. & Chang, B. J. (2007) Microbiology 153, 676-85.
33. Rutherford, K., J. Parkhill, J. Crook, T. Horsnell, P. Rice, M-A. Rajandream and B. Barrell. (2000) Bioinformatics 16, 944-945.
34. Altschul, S. F., Thomas L. Madden, Alejandro A. Schaffer, & Jinghui Zhang, Z. Z., Webb Miller, and David J. Lipman (1997) Nucleic Acids Res. 25, 3389-3402
35. Schaffer, A. A., L. Aravind, Thomas L. Madden, Sergei, Shavirin, J. L. S., Yuri I. Wolf, Eugene V. Koonin, and & Altschul, S. F. (2001) Nucleic Acids Res. 29, 2994-3005.
36. Sebaihia, M., Wren, B. W., Mullany, P., Fairweather, N. F., Minton, N., Stabler, R., Thomson, N. R., Roberts, A. P., Cerdeno-Tarraga, A. M., Wang, H., Holden, M. T., Wright, A., Churcher, C., Quail, M. A., Baker, S., Bason, N., Brooks, K., Chillingworth, T., Cronin, A., Davis, P., Dowd, L., Fraser, A., Feltwell, T., Hance, Z., Holroyd, S., Jagels, K., Moule, S., Mungall, K., Price, C., Rabbinowitsch, E., Sharp, S., Simmonds, M., Stevens, K., Unwin, L., Whithead, S., Dupuy, B., Dougan, G., Barrell, B. & Parkhill, J. (2006) Nat Genet. 38, 779-86.
37. Loessner, M. J., Wendlinger, G. & Scherer, S. (1995) Mol Microbiol 16, 1231-41.
38. Wegmann, U., Klein, J. R., Drumm, I., Kuipers, O. P. & Henrich, B. (1999) Appl Environ Microbiol 65, 4729-33.
39. Mayer et al., 2008, J. Bacteriol. 190:6734-6740.
40. Fernandez, A., Horn, N., Wegmann, U., Nicoletti, C., Gasson, M. J. and Narbad, A. (2009). Appl Environ Microbiol 75, 869-71.
41. Qazi, S. N. A., Counil, E., Morrissey, J., Rees, C. E. D., Cockayne, A., Winzer, K., Chan, W. C., Williams, P. and Hill, P. P (2001). Infection and Immunity 69, 7074-7082.

1-100. (canceled)

101. An isolated polypeptide comprising a fragment of the amino acid sequence of SEQ ID NO:1, or a variant, derivative or fusion thereof, wherein the polypeptide is capable of binding specifically to and lysing cells of Clostridium difficile and wherein the polypeptide exhibits greater lytic activity on cells of Clostridium difficile than the polypeptide of SEQ ID NO:1.

102. The isolated polypeptide of claim 101, wherein the polypeptide exhibits greater lytic activity on cells of Clostridium difficile ribotype 027 than the polypeptide of SEQ ID NO:1.

103. The isolated polypeptide of claim 101, wherein the polypeptide exhibits at least 110% of the lytic activity of the polypeptide of SEQ ID NO:1 on cells of Clostridium difficile, for example at least 120%, 130%, 140%, 150%, 160%, 170%, 180%, 200%, 250%, 300%, 400%, or 500%.

104. The isolated polypeptide of claim 101, wherein the polypeptide is at least 50 amino acids in length, for example at least 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, or 500 amino acids in length.

105. The isolated polypeptide of claim 101, wherein the fragment corresponds to amino acids 1 to 179 of SEQ ID NO:1 (i.e., SEQ ID NO:2).

106. The isolated polypeptide of claim 101, comprising or consisting of a fragment of the amino acid sequence of SEQ ID NO:1.

107. The isolated polypeptide of claim 101, comprising a variant that comprises or consists of an amino acid sequence with at least 60% identity to the amino acid sequence of SEQ ID NO:1, or to a fragment thereof, more preferably at least 70% or 80% or 85% or 90% identity to said sequence, and most preferably at least 95%, 96%, 97%, 98% or 99% identity to said amino acid sequence.

108. The isolated polypeptide of claim 101, consisting of the amino acid sequence of SEQ ID NO:2, 4, or 6.

109. An isolated nucleic acid molecule encoding the polypeptide of claim 101.

110. A vector comprising a nucleic acid molecule encoding the polypeptide of claim 101.

111. A host cell comprising a nucleic acid molecule encoding the polypeptide of claim 101 or a vector comprising a nucleic acid molecule encoding the polypeptide of claim 101.

112. A method for producing the polypeptide of claim 101 comprising culturing a population of host cells comprising a nucleic acid molecule encoding the polypeptide of claim 101 or a vector comprising a nucleic acid molecule encoding the polypeptide of claim 101 under conditions in which the polypeptide is expressed, and isolating the polypeptide therefrom.

113. A pharmacological composition comprising:
(a) the polypeptide of claim 101;
(b) a nucleic acid molecule encoding the polypeptide of claim 101;
(c) a vector comprising a nucleic acid molecule encoding the polypeptide of claim 101;
(d) a host cell comprising a nucleic acid molecule encoding the polypeptide of claim 101 or a vector comprising a nucleic acid molecule encoding the polypeptide of claim 101; and/or
(e) a bacteriophage capable of expressing the polypeptide of claim 101; and
a pharmaceutically acceptable carrier, diluent, or excipient.

114. A method for killing and/or inhibiting/preventing the growth of microbial cells in a patient, the method comprising administering to the patient the polypeptide of claim 101, or a nucleic acid molecule, vector, host cell, or bacteriophage capable of expressing the same, wherein the microbial cells are selected from the group consisting of Clostridium difficile cells and other bacterial cells susceptible to lysis upon contact with a polypeptide of SEQ ID NO:1.

115. A method for the treatment or prevention a disease or condition associated with microbial cells in a patient, the method comprising administering to the patient the polypeptide of claim 101, or a nucleic acid molecule, vector, host cell, or bacteriophage capable of expressing the same, wherein the microbial cells are selected from the group consisting of Clostridium difficile cells and other bacterial cells susceptible to lysis upon contact with a polypeptide of SEQ ID NO:1.

116. Use of the polypeptide of claim 101, or a nucleic acid molecule, vector, host cell, or bacteriophage capable of expressing the same, for killing and/or inhibiting/preventing the growth of microbial cells in vitro and/or ex vivo, wherein the microbial cells are selected from the group consisting of Clostridium difficile cells and other bacterial cells susceptible to lysis upon contact with a polypeptide of SEQ ID NO:1.

117. A kit for detecting the presence of microbial cells in a sample, the kit comprising the polypeptide of claim 101, or a nucleic acid molecule, vector, host cell, or bacteriophage capable of expressing the same, wherein the microbial cells are selected from the group consisting of Clostridium difficile cells and other bacterial cells susceptible to lysis upon contact with a polypeptide of SEQ ID NO:1.

118. Use of the polypeptide of claim 101, or a nucleic acid molecule, vector, host cell, or bacteriophage capable of expressing the same, in the preparation of a diagnostic agent for a disease or condition associated with microbial cells selected from the group consisting of Clostridium difficile cells and other bacterial cells susceptible to lysis upon contact with a polypeptide of SEQ ID NO:1.

119. The polypeptide of claim 101, or a nucleic acid molecule, vector, host cell, or bacteriophage capable of expressing the same, for use in the diagnosis of a disease or condition associated with microbial cells selected from the group consisting of Clostridium difficile cells and other bacterial cells susceptible to lysis upon contact with a polypeptide of SEQ ID NO:1.

120. Use of the polypeptide of claim 101, or a nucleic acid molecule, vector, host cell, or bacteriophage capable of expressing the same, for detecting the presence of microbial cells in a sample in vitro and/or ex vivo, wherein the microbial cells selected from the group consisting of Clostridium difficile cells and other bacterial cells susceptible to lysis upon contact with a polypeptide of SEQ ID NO:1.


SEQUENCE LISTING (29 sequences)

SEQ ID NO: 1 - protein, 270 aa, Clostridium phage phiCD27
MKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNINNEGVKQMYKHTIVYDGEVDKISATVVGWGYNDGKILICDIKDYVPGQTQNLYVVGGGACEKISSITKEKFIMIKGNDRFDTLYKALDFINR

SEQ ID NO: 2 - protein, 179 aa, Artificial Sequence
  CD27L1-179
MKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNIN

SEQ ID NO: 3 - protein, 20 aa, Artificial Sequence
  6-His tag
MGSSHHHHHHSSGLVPRGSH

SEQ ID NO: 4 - protein, 199 aa, Artificial Sequence
  6-His tagged, truncated phiCD27 lysin
MGSSHHHHHHSSGLVPRGSHMKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNIN

SEQ ID NO: 5 - protein, 9 aa, Artificial Sequence
  6-His tag
MSHHHHHHA

SEQ ID NO: 6 - protein, 188 aa, Artificial Sequence
  6-His tagged, truncated phiCD27 lysin
MSHHHHHHAMKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNIN

SEQ ID NO: 7 - DNA, 813 bp, Clostridium phage phiCD27
atgaaaatatgtataacagtaggacacagtattttaaaaagtggagcatgtacttctgctgatggagtagttaacgagtatcaatacaacaaatctcttgcaccagtattagcagatacatttagaaaagaagggcataaggtagatgtaataatatgcccagaaaagcagtttaaaactaagaatgaagaaaagtcttataaaatacctagagttaatagtggaggatatgatttacttatagagttacatttaaatgcaagtaacggtcaaggtaaaggttcagaagtcctatattatagtaataaaggcttagagtatgcaactagaatatgtgataaactaggtacagtatttaaaaatagaggtgctaaattagataaaagattatatatcttaaatagttcaaagcctacagcagtattaattgaaagtttcttctgtgataataaagaagattatgataaagctaagaaactaggtcatgaaggtattgctaagttaattgtagaaggtgtattaaataaaaatataaataatgagggagttaaacagatgtacaaacatacaattgtttatgatggagaagttgacaaaatctctgcaactgtagttggttggggttataatgatgggaaaatactgatatgtgatataaaagattacgtgccaggtcagacgcaaaatctttatgttgtaggaggtggcgcatgtgaaaagataagttctattactaaagaaaaatttattatgataaaaggtaatgatagatttgatacactttataaagcattggattttattaatagatag

SEQ ID NO: 8 - DNA, 29 bp, Artificial Sequence
  PCR primer CD27L_NDE
ttacatatgaaaatatgtataacagtagg

SEQ ID NO: 9 - DNA, 21 bp, Artificial Sequence
  PCR primer CD27L_XHO
caaccacctcgagttgataac

SEQ ID NO: 10 - DNA, 27 bp, Artificial Sequence
  PCR primer CD630L1_NDE
tgctcatatgaaaataggtataaattg

SEQ ID NO: 11 - DNA, 17 bp, Artificial Sequence
  PCR primer M13 forward
gtaaaacgacggccagt

SEQ ID NO: 12 - DNA, 23 bp, Artificial Sequence
  oligomer encoding a 6-histidine tag
agtcatcaccatcaccatcacgc

SEQ ID NO: 13 - DNA, 24 bp, Artificial Sequence
  PCR primer CD27LCOD2_F
aaaatatgtataacagtaggacac

SEQ ID NO: 14 - DNA, 25 bp, Artificial Sequence
  PCR primer LM4Nde
ggatgattacatatggcattaacag

SEQ ID NO: 15 - DNA, 30 bp, Artificial Sequence
  PCR primer LM4-splice-CD27LE
tatacatattttcatgttttgtgtcgcagt

SEQ ID NO: 16 - DNA, 30 bp, Artificial Sequence
  PCR primer LM4-splice-CD27
tttaactccctcattgttttgtgtcgcagt

SEQ ID NO: 17 - DNA, 19 bp, Artificial Sequence
  PCR primer T7T
gctagttattgctcagcgg

SEQ ID NO: 18 - DNA, 35 bp, Artificial Sequence
  PCR primer CD27LEspliceLM4
actgcgacacaaaacatgaaaatatgtataacagt

SEQ ID NO: 19 - DNA, 30 bp, Artificial Sequence
  PCR primer CD27LspliceLM4
ctgcgacacaaaacaatgagggagttaaac

SEQ ID NO: 20 - DNA, 864 bp, Listeria phage phiLM4
atggcattaacagaggcatggctaattgaaaaagcaaatcgcaaattgaatacgtcaggtatgaataaagctacatctgataagactcggaatgtaattaaaaaaatggcaaaagaagggatttatctttgtgttgcgcaaggttaccgctcaacagcggaacaaaatgcgctatatgcacaagggagaaccaaacctggagcgattgttactaatgctaaaggtgggcaatctaatcataatttcggtgtagcagttgatttgtgcttgtatacgagcgacggaaaagatgttatttgggagtcgacaacttcccggtggaaaaaggttgttgctgctatgaaagcggaaggattcgaatggggcggagattggaaaagttttaaagactatccgcattttgaactatgtgacgctgtaagtggtgagaaaatccctactgcgacacaaaacaccaatccaaacagacatgatgggaaaatcgttgacagcgcgccactattgccaaaaatggactttaaatcaaatccagcgcgcatgtataaatcaggaactgagttcttagtatatgaacataatcaatattggtacaagacgtacatcaacgacaaattatactacatgtataagagcttttgcgatgttgtagctaaaaaagatgcaaaaggacgcatcaaagttcgaattaaaagcgcgaaagacttacgaattccagtttggaataacacaaaattgaattctgggaaaattaaatggtatgcacccaatacaaaattagcatggtacaacaacggaaaaggatacttggaactctggtatgaaaaggatggctggtactacacagcgaactacttcttaaaataa

SEQ ID NO: 21 - protein, 287 aa, Listeria phage phiLM4
MALTEAWLIEKANRKLNTSGMNKATSDKTRNVIKKMAKEGIYLCVAQGYRSTAEQNALYAQGRTKPGAIVTNAKGGQSNHNFGVAVDLCLYTSDGKDVIWESTTSRWKKVVAAMKAEGFEWGGDWKSFKDYPHFELCDAVSGEKIPTATQNTNPNRHDGKIVDSAPLLPKMDFKSNPARMYKSGTEFLVYEHNQYWYKTYINDKLYYMYKSFCDVVAKKDAKGRIKVRIKSAKDLRIPVWNNTKLNSGKIKWYAPNTKLAWYNNGKGYLELWYEKDGWYYTANYFLK

SEQ ID NO: 22 - DNA, 1266 bp, Artificial Sequence
  Domain-swapped lysin LM4-CD27LE
atggcattaacagaggcatggctaattgaaaaagcaaatcgcaaattgaatacgtcaggtatgaataaagctacatctgataagactcggaatgtaattaaaaaaatggcaaaagaagggatttatctttgtgttgcgcaaggttaccgctcaacagcggaacaaaatgcgctatatgcacaagggagaaccaaacctggagcgattgttactaatgctaaaggtgggcaatctaatcataatttcggtgtagcagttgatttgtgcttgtatacgagcgacggaaaagatgttatttgggagtcgacaacttcccggtggaaaaaggttgttgctgctatgaaagcggaaggattcgaatggggcggagattggaaaagttttaaagactatccgcattttgaactatgtgacgctgtaagtggtgagaaaatccctactgcgacacaaaacatgaaaatatgtataacagtaggacacagtattttaaaaagtggagcatgtacttctgctgatggagtagttaacgagtatcaatacaacaaatctcttgcaccagtattagcagatacatttagaaaagaagggcataaggtagatgtaataatatgcccagaaaagcagtttaaaactaagaatgaagaaaagtcttataaaatacctagagttaatagtggaggatatgatttacttatagagttacatttaaatgcaagtaacggtcaaggtaaaggttcagaagtcctatattatagtaataaaggcttagagtatgcaactagaatatgtgataaactaggtacagtatttaaaaatagaggtgctaaattagataaaagattatatatcttaaatagttcaaagcctacagcagtattaattgaaagtttcttctgtgataataaagaagattatgataaagctaagaaactaggtcatgaaggtattgctaagttaattgtagaaggtgtattaaataaaaatataaataatgagggagttaaacagatgtacaaacatacaattgtttatgatggagaagttgacaaaatctctgcaactgtagttggttggggttataatgatgggaaaatactgatatgtgatataaaagattacgtgccaggtcagacgcaaaatctttatgttgtaggaggtggcgcatgtgaaaagataagttctattactaaagaaaaatttattatgataaaaggtaatgatagatttgatacactttataaagcattggattttattaatagatag

SEQ ID NO: 23 - protein, 421 aa, Artificial Sequence
  Domain-swapped lysin LM4-CD27LE
MALTEAWLIEKANRKLNTSGMNKATSDKTRNVIKKMAKEGIYLCVAQGYRSTAEQNALYAQGRTKPGAIVTNAKGGQSNHNFGVAVDLCLYTSDGKDVIWESTTSRWKKVVAAMKAEGFEWGGDWKSFKDYPHFELCDAVSGEKIPTATQNMKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNINNEGVKQMYKHTIVYDGEVDKISATVVGWGYNDGKILICDIKDYVPGQTQNLYVVGGGACEKISSITKEKFIMIKGNDRFDTLYKALDFINR

SEQ ID NO: 24 - DNA, 729 bp, Artificial Sequence
  Domain-swapped lysin LM4-CD27L
atggcattaacagaggcatggctaattgaaaaagcaaatcgcaaattgaatacgtcaggtatgaataaagctacatctgataagactcggaatgtaattaaaaaaatggcaaaagaagggatttatctttgtgttgcgcaaggttaccgctcaacagcggaacaaaatgcgctatatgcacaagggagaaccaaacctggagcgattgttactaatgctaaaggtgggcaatctaatcataatttcggtgtagcagttgatttgtgcttgtatacgagcgacggaaaagatgttatttgggagtcgacaacttcccggtggaaaaaggttgttgctgctatgaaagcggaaggattcgaatggggcggagattggaaaagttttaaagactatccgcattttgaactatgtgacgctgtaagtggtgagaaaatccctactgcgacacaaaacaatgagggagttaaacagatgtacaaacatacaattgtttatgatggagaagttgacaaaatctctgcaactgtagttggttggggttataatgatgggaaaatactgatatgtgatataaaagattacgtgccaggtcagacgcaaaatctttatgttgtaggaggtggcgcatgtgaaaagataagttctattactaaagaaaaatttattatgataaaaggtaatgatagatttgatacactttataaagcattggattttattaatagatag

SEQ ID NO: 25 - protein, 240 aa, Artificial Sequence
  Domain-swapped lysin LM4-CD27L
MALTEAWLIEKANRKLNTSGMNKATSDKTRNVIKKMAKEGIYLCVAQGYRSTAEQNALYAQGRTKPGAIVTNAKGGQSNHNFGVAVDLCLYTSDGKDVIWESTTSRWKKVAAMKAEGFEWGGDWKSFKDYPHFELCDAVSGEKIPTATQNNEGVKQMYKHTIVYDGEVDKISATVVGWGYNDGKILICDIKDYVPGQTQNLYVVGGGACEKISSITKEKFIMKGNDRFDTLYKALDFINR

SEQ ID NO: 26 - protein, 92 aa, Artificial Sequence
  CD27L180-270
MNEGVKQMYKHTIVYDGEVDKISATVVGWGYNDGKILICDIKDYVPGQTQNLYVVGGGACEKISSITKEKFIMIKGNDRFDTLYKALDFINR

SEQ ID NO: 27 - DNA, 20 bp, Artificial Sequence
  PCR primer T7P
taatacgactcactataggg

SEQ ID NO: 28 - DNA, 23 bp, Artificial Sequence
  PCR primer CD27L EAD
ttaactccctcctaatttatatt

SEQ ID NO: 29 - DNA, 27 bp, Artificial Sequence
  PCR primer CD27L_CBD
catatgaatgagggagttaaacagatg